(19) United States
(12) Patent Application Publication
(10) Pub. No.: US 2014/0105993 A1
(43) Pub. Date: Apr. 17, 2014

(54) MODIFIED-RELEASE MICROPARTICLES BASED ON AMPHIPHILIC COPOLYMER AND ON ACTIVE PRINCIPLE(S) AND PHARMACEUTICAL FORMULATIONS COMPRISING THEM

(71) Applicant: Flamel Technologies, S.A., Venissieux Cedex (FR)

(72) Inventors: Alain Constancis, Lyon (FR); You-Ping Chan, Ternay (FR)

(73) Assignee: Flamel Technologies, S.A., Venissieux Cedex (FR)

(21) Appl. No.: 14/086,635

(22) Filed: Nov. 21, 2013

Related U.S. Application Data
(63) Continuation of application No. 11/878,947, filed on Jul. 27, 2007, now abandoned.
(60) Provisional application No. 60/833,778, filed on Jul. 28, 2006.

(51) Int. Cl. A61K 47/42; A61K 9/16; A61K 38/28; A61K 38/27; A61K 38/21; A61K 38/20

(52) U.S. Cl. CPC A61K 47/42; A61K 38/212; A61K 38/2013; A61K 38/28; A61K 38/27; A61K 9/1658
USPC 424/492; 424/85.7; 424/85.2; 514/5.9; 514/11.4; 424/499; 424/490; 424/494; 424/497

(57) ABSTRACT

The present invention relates to novel microparticles formed of amphiphilic polyamino acids which transport active principle(s), AP(s), in particular protein and peptide active principle(s), and to novel modified-release pharmaceutical formulations comprising said AP microparticles. Microparticles of amphiphilic polyamino acid (PO) may include at least one AP (associated noncovalently) which spontaneously form a colloidal suspension of nanoparticles in water, at pH 7.0, under isotonic conditions. The microparticles may be obtained by atomization of a solution or colloidal suspension of PO comprising at least one AP, may have a size of between 0.5 and 100 microns, and may be dispersible in colloidal suspension.

### CLAIM OF PRIORITY

This application is a continuation of U.S. patent application Ser. No. 11/878,947, filed Jul. 27, 2007, which claims priority to U.S. Provisional Application No. 60/833,778, filed Jul. 28, 2006. The contents each are incorporated herein by reference in their entirety.

### FIELD OF THE INVENTION

The present invention relates to novel transporters of active principle(s) (APs), in particular protein and peptide active principle(s), and to novel modified-release pharmaceutical formulations comprising said AP transporters. There are many therapeutic (human and veterinary) applications of these formulations.

The reference AP used throughout the present account targets at least one active principle. The term “modified release” denotes a prolonged or delayed or pulsatile release. More specifically, the novel AP transporters targeted by the invention are microparticles formed of amphiphilic polymers, for example polyamino acids modified by hydrophobic groups. These microparticles comprise at least one AP associated with the polymer and can be provided in the form of colloidal suspensions or in the dry form.

### CONTEXT OF THE INVENTION

In the field of the prolonged release of pharmaceutical APs, in particular therapeutic peptides and proteins, the aim is very often to reproduce as much as possible in the patient a plasma peptide or protein concentration close to the value observed in the healthy subject.

This objective conflicts with the low lifetime of proteins in plasma, which results in the therapeutic protein being repeatedly injected. The plasma concentration of therapeutic protein then exhibits a sawtooth profile characterized by high concentration peaks and very low concentration minima. The concentration peaks, which are much higher than the basal concentration in the healthy subject, have very marked harmful effects due to the high toxicity of therapeutic proteins, such as interleukin IL-2. Furthermore, the concentration minima are below the concentration necessary to have a therapeutic effect, which results in poor therapeutic coverage of the patient and serious long-term consequences.

Consequently, in order to reproduce in the patient a plasma concentration of therapeutic protein close to the ideal value for the treatment of the patient, it is important for the pharmaceutical formulation under consideration to make it possible to release the therapeutic protein over a prolonged period of time, so as to limit the variations in plasma concentration over time.

Furthermore, this active formulation should preferably satisfy the following specifications, already known to a person skilled in the art:
1—prolonged release of an active and nondenatured therapeutic protein, for example a human or synthetic protein, so that the plasma concentration is maintained at the therapeutic level;
2—sufficiently low viscosity of the formulation to be easily injectable;
3—biocompatible and biodegradable form;
4—form not exhibiting toxicity or immunogenicity;
5—form having excellent local tolerance.

In attempting to achieve these objectives, one of the best approaches provided in the prior art was to develop prolonged-release forms of therapeutic protein(s) composed of low-viscosity liquid suspensions of nanoparticles charged with therapeutic proteins. These suspensions have made possible the ready administration of native therapeutic proteins.

Thus, the therapeutic protein has been associated with nanoparticles of a copolyamino acid comprising hydrophobic groups and hydrophilic groups.

U.S. Pat. No. 5,904,936 discloses submicron-sized particles (NPV), with a mean size of between 0.01 and 0.5 μm, and micron-scale (micron-sized) particles (MPV), with a mean size of between 0.5 and 20 μm, of amphiphilic copolymer of polyamino acids comprising at least two types of amino acids, one being neutral and hydrophobic and the other being ionizable. Proteins, such as insulin, are spontaneously adsorbed in aqueous solution at these particles. The polyamino acid copolymer is, for example, a poly(L-leucine-b-(sodium L-glutamate)) block copolymer. This patent discloses the aggregation of NPV to give MPV by addition to a colloidal suspension of poly-Leu/Glu of monocationic salts (ammonium sulfate), polycationic salts (Fe²⁺, Fe³⁺, Zn²⁺, Ca²⁺, Al²⁺, Al³⁺ or Cu²⁺), of acid (HCl) or of cationic polymers (polylysine).

Patent application WO-A-03/104303 discloses amphiphilic polyamino acids comprising aspartic residues or glutamic residues, at least a portion of these residues carrying grafts comprising at least one alpha-tocopherol unit, (for example: polyglutamate or polyaspartate grafted by alpha tocopherol of synthetic or natural origin). These hydrophobically modified homopolyamino acids spontaneously form, in water, a colloidal suspension of nanoparticles which are capable of easily associating in aqueous suspension at pH=7.4 with at least one active protein (insulin).

The duration of release in vivo of the active protein(s) (for example insulin) vectorized by the suspensions according to U.S. Pat. No. 5,904,936 or WO-A-2003/104303 could do with being increased.

The increase in the duration of release was partially obtained by the pharmaceutical forms disclosed in PCT application WO-A-05/051416. This application discloses a colloidal suspension of nanoparticles (0.001-0.5 μm) of hydrophobically modified poly(sodium L-glutamate) which is used at a concentration such that, after subcutaneous injection, a gel is formed in situ in the patient on contact with the endogenous albumin. The protein is then slowly released over a period typically of one week. However, when the concentration of therapeutic protein to be administered is relatively high, as is the case, for example, for human growth hormone, the duration of release is limited to only a few days.

### OBJECTS OF THE INVENTION

It is desirable to improve this prior art by providing a pharmaceutical formulation for the prolonged release of AP:
which makes it possible, after injection by the parenteral route (for example, subcutaneously), to obtain a prolonged duration of release in vivo for APs (for example, therapeutic proteins, therapeutic peptides and small molecules) which are nondenatured and highly concentrated, for example at several mg/ml,
and which would be stable on storage, both physicochemically and biologically.

To do this, it is advisable to aggregate together the nanoparticles of the formulation according to WO 05/051416 to give microparticles, by means of polyvalent ions of opposite polarity to that of the ionizable groups (IG) of the amphiphilic polyamino acid, said ions being present in well defined proportions. This results in a selection of a specific population of microparticles which make possible a significant extension in the duration of release of the AP (for example, protein or peptide) with which they are combined. Some polyvalent ions, such as Mg²⁺, Ca²⁺, Zn²⁺, Fe²⁺, Cu²⁺ or their mixtures or Al³⁺, Fe³⁺ or their mixtures, confer excellent tolerance on such a liquid pharmaceutical formulation for the prolonged release of AP.

This formulation can, for example, comprise an aqueous colloidal suspension of low viscosity based on micron-scale particles of polyglutamate grafted by alpha-tocopherol of synthetic origin with a size of between 0.5 and 100 μm, comprising polyvalent ions Mg²⁺, Ca²⁺, Zn²⁺, Fe²⁺, Cu²⁺, Al³⁺ or Fe³⁺, with a ratio r

corresponding to the formula

$$r = n \times \frac{[ PI ]}{[ IG ]} ,$$

where
n is the valency of said polyvalent ions,
[PI] is the molar concentration of polyvalent ions,
[IG] is the molar concentration of ionizable groups IG,
   of between 0.3 and 10.

These selected micron-scale particles result from the aggregation of a large number of nanoparticles of amphiphilic copolymer.

A suspension of microparticles charged with AP (for example hGH) can thus be manufactured by flocculation with polyvalent ions Mg²⁺, Ca²⁺, Zn²⁺, Fe²⁺, Cu²⁺, Al³⁺ or Fe³⁺, this flocculation being followed by maturing and washing. The suspension can subsequently be lyophilized or atomized and then reconstituted with water to produce a formulation ready for injection.

It is clear that these dry powder forms of microparticles are a priori advantageous with regard to storage. Specifically, they should make it possible to increase the physical stability of these microparticles and the stability of the AP.

However, in the context of a therapeutic use of these microparticle powders by the parenteral route, the difficulty consists in being able to easily disperse (or suspend) these powders at the time of use in order to obtain a reconstituted liquid microparticle form, more specifically a suspension, which is stable, for example at least for a few minutes, indeed even for at least a few tens of minutes, at ambient temperature. This stability is desired in particular in order to make possible easy handling of this reconstituted suspension. It is also important for this reconstituted suspension to be able to be easily injected by passing through a hollow needle in combination with a syringe or with an injection pen (insulin pen type). Finally, it is advisable to take into account the fact that the operations of dispersing, handling and injecting the reconstituted suspension are intended to be carried out by patients or medical personnel.

In this context, one of the essential objects of the invention would be to provide novel microparticles obtained by aggregation of nanoparticles of biodegradable and water-soluble amphiphilic polymer, for example of the type of those according to the formulation disclosed in WO-A-05/051416, these microparticles being charged with AP and being capable of exhibiting improved properties, in particular in the dry solid form, especially with regard to their ability to be dispersed (or dispersibility) and, with regard to the reconstituted suspension, its stability and its ability to be easily handled and injected.

Another object of the invention is to provide novel microparticles obtained by aggregation of nanoparticles of biodegradable and water-soluble amphiphilic polymer, said microparticles being charged with AP and having good dispersing properties, whether in the aqueous phase or in the organic phase, while retaining the integrity of said microparticles.

Another object of the invention is to provide novel microparticles obtained by aggregation of nanoparticles of biodegradable and water-soluble amphiphilic polymer, said microparticles being charged with AP and having excellent stability properties in the solid and dry form.

Another object of the invention is to provide a novel process for the preparation of microparticles in the solid and dry form, these microparticles furthermore being:
obtained by aggregation of nanoparticles of biodegradable and water-soluble amphiphilic polymer, for example of the type of those according to the formulation disclosed in WO-A-05/051416,
charged with AP,
and as defined in the above objects.

Another object of the invention is to provide a process for the preparation of microparticles in the solid and dry form of the type of that targeted in the above object and which is, furthermore, simple, economic and industrial.

Another object of the invention is to provide a pharmaceutical formulation comprising novel microparticles obtained by aggregation of nanoparticles of biodegradable and water-soluble amphiphilic polymer, for example of the type of those according to the formulation disclosed in WO-A-03/104303, these microparticles furthermore being:
charged with AP,
and as defined in the above objects.

Another object of the invention is to provide a pharmaceutical formulation for the prolonged release of AP which overcomes the deficiencies of the prior art and in particular which makes it possible, after injection by the parenteral route (for example, subcutaneously), to obtain a prolonged duration of release in vivo of nondenatured APs (for example, therapeutic proteins, therapeutic peptides or small molecules).

Another object of the invention is to provide a pharmaceutical formulation which makes it possible, after injection by the parenteral route (for example, subcutaneously), to obtain a prolonged duration of release in vivo of highly concentrated therapeutic proteins or peptides, for example at several mg/ml.

Another object of the invention is to provide a pharmaceutical formulation for prolonged release of the AP in vivo which is stable on storage, both physicochemically and biologically.

Another object of the invention is to provide a pharmaceutical formulation for prolonged release of the AP in vivo which exhibits at least one of the following properties: biocompatibility, biodegradability, nontoxicity and good local tolerance.

Another object of the invention is to provide a pharmaceutical formulation for the slow prolonged release of an AP in vivo comprising microparticles of amphiphilic polymer PO which are self-associated with at least one AP (PO/AP microparticles), the polymer PO being a water-soluble biodegradable polymer carrying hydrophobic groups (HG) and hydrophilic groups (preferably ionizable groups (IG) at least partially ionized), spontaneously forming in water a suspension of colloidal nanoparticles, this polymer PO being, for example, a polyamino acid in which the main chain is formed by aspartic residues or glutamic residues, at least a portion of these residues being modified by grafting at least one hydrophobic group HG in the chain or at the chain end.

Another object of the invention is to provide a kit for reconstituting the formulation as defined in the objects set out above, this kit being, for example, simple to use so that it can be easily employed by the patient or by the medical personnel.

Another object of the invention is to provide a process for reconstituting the formulation as defined in the objects set out above, this process being, for example, simple to carry out, in particular by the patient or the medical personnel.

Another object of the invention is to provide a solid pharmaceutical formulation for the prolonged release of AP, in particular a dry powder form for pulmonary inhalation and administration:
based on PO microparticles associated with at least one AP, as are defined in the objects set out above; or
obtained from the formulation as defined in the objects set out above.

### BRIEF DESCRIPTION OF THE INVENTION

In order to achieve these objects, among others, the inventors have had the credit of discovering, after lengthy and laborious research, that, in an entirely surprising and unexpected way, the atomization of formulations based on amphiphilic PO (for example, on copolyamino acids) and on AP results in dry PO/AP microparticles which are very stable and which make it possible to reconstitute aqueous suspensions of microparticles in a liquid medium which have a size of between 0.5 and 100 μm.

Furthermore, the inventors have had the credit of developing means for reconstituting a suspension from these microparticles, said means making it possible to optimize their dispersion both in an aqueous liquid phase and in an organic liquid phase.

This is because a stable easy dispersion of good quality is a precondition for the use of these suspensions, reconstituted from dry PO/AP microparticles, as injectable formulations.

Atomization is an industrial technique known for producing dry particles from a solution or suspension of the constituent material of said particles. Atomization or spray drying consists in very rapidly evaporating, in a stream of hot air or hot inert gas, nebulized droplets of this solution or suspension.

In the pharmaceutical field, atomization can be problematic as it imposes a heat stress which can prove to be absolutely unacceptable for some heat-sensitive APs, such as APs based on proteins or other peptide compounds. Recourse to atomization to produce dry particles based on excipients and on AP is therefore not obvious, except by choosing excipients capable of preventing or of minimizing the denaturation of peptide APs.

Thus it is that application US-A-2005/0158392 discloses the preparation by atomization of solid lipophilic microparticles charged with peptide AP. This atomization consists either in atomizing an aqueous solution comprising hyaluronic acid, AP and a lipophilic surface-active agent (for example, lecithin), indeed even another surfactant of the Tween® 80 type, or, in a first step, in atomizing an aqueous solution comprising hyaluronic acid, AP and optionally a surfactant of the Tween® 80 type, in order to obtain primary particles, and, in a second step, in atomizing an alcoholic solution of lipophilic surface-active agent (for example, lecithin) in which the primary particles are dispersed. The accent in this US application is placed on the protective combination of hyaluronic acid and lipophilic surface-active agent (for example, lecithin), the latter being intended to form a coating film for the microparticles based on hyaluronic acid and on AP. This document neither mentions nor even alludes to the use of amphiphilic POs and even less of amphiphilic copolyamino acids as excipients or transporters of AP together forming the microparticles.

Another atomization example is that described in the paper by Maa et al., J. Pharm. Sci., 87(2), 152-159, 1988. According to this document, the AP (human growth hormone) is complexed by zinc in the presence of biodegradable surfactants before being atomized to give microparticles.

It is thus apparent that the atomization of proteins is a difficult operation requiring the use of complex formulations comprising numerous excipients present to protect the AP during the process and to guarantee its stability on storage, which is particularly crucial for certain sensitive molecules, such as human growth hormone (hGH).

Furthermore, the credit to the inventors is all the greater as the prior art teaches nothing with regard to the difficult problem of the dispersion and of the stabilization in a liquid of dry microparticles (in particular with regard to microparticles of amphiphilic PO) designed for the prolonged release of AP.

It follows from this that the invention relates, in a first aspect, to microparticles of polymer (PO) comprising at least one active principle (AP), the polymer PO:
being a water-soluble biodegradable amphiphilic copolymer carrying hydrophobic groups (HG) and hydrophilic groups,
spontaneously forming a colloidal suspension of nanoparticles in water, at pH=7.0, under isotonic conditions,
and being associated noncovalently with the AP;
   which microparticles:
a. are obtained by atomization of a solution or colloidal suspension of PO comprising at least one AP,
b. have a size, measured in a T test, of between 0.5 and 100 μm, preferably between 1 and 70 μm, preferably between 2 and 40 μm,
c. and are dispersible in colloidal suspension in a DP1 dispersibility test.

In a second aspect, the invention relates to a process for the preparation of microparticles of polymer PO associated with at least one active principle (AP), these PO/AP microparticles being in particular those defined above according to the first aspect of the invention,

i. the polymer PO:
being a water-soluble biodegradable amphiphilic copolymer carrying hydrophobic groups (HG) and hydrophilic groups (preferably ionizable groups (IG) at least partially ionized),
spontaneously forming a colloidal suspension of nanoparticles in water, at pH=7.0, under isotonic conditions,
and being associated noncovalently with the AP;
   ii. said microparticles having a size, measured in a T test, of between 0.5 and 100 μm, preferably between 1 and 70 μm, preferably between 2 and 40 μm,
   which comprises essentially atomizing a solution or a colloidal suspension of PO comprising AP.

According to an advantageous alternative form, the microparticles obtained by atomization are redispersed in an essentially aqueous liquid medium (preferably comprising polyvalent ions as dispersing means) and then the dispersion obtained is lyophilized.

In a third aspect, the invention relates to a liquid pharmaceutical formulation for the prolonged release of AP, which comprises a colloidal suspension, of low viscosity, based on PO microparticles comprising at least one AP, these microparticles being those defined above according to the first aspect of the invention or those obtained by the process defined above according to the second aspect of the invention.

In a fourth aspect, the invention relates to a reconstitution kit, in particular for reconstituting the formulation as defined above according to a third aspect of the invention, which comprises:
PO microparticles comprising at least one AP, these microparticles being those defined above according to the first aspect of the invention or those obtained by the process defined above according to the second aspect of the invention;
and a reconstitution liquid chosen from the group consisting of:
   essentially aqueous liquids;
   essentially organic and water-miscible liquids;
   and essentially organic water-immiscible liquids.

In a fifth aspect, the invention relates to a reconstitution process, in particular for reconstituting the formulation as defined above according to a third aspect of the invention, which comprises essentially:
mixing:
   [symbol] PO microparticles comprising at least one AP, these microparticles being those defined above according to the first aspect of the invention or those obtained by the process defined above according to the second aspect of the invention;
   [symbol] and a reconstitution liquid chosen from the group consisting of:
   →essentially aqueous liquids;
   →essentially organic and water-miscible liquids;
   →and essentially organic water-immiscible liquids;
and stirring this mixture.

In a sixth aspect, the invention relates to a solid pharmaceutical formulation for the prolonged release of AP, which comprises a dry powder form for inhalation and pulmonary administration:
based on PO microparticles comprising at least one AP, these microparticles being those defined above according to the first aspect of the invention or those obtained by the process defined above according to the second aspect of the invention;
or obtained from the formulation as defined above according to a third aspect of the invention.

Advantages:

Without wishing to be committed to a theory, it may be believed that the hydrophobic groups of the polymers PO, which can assemble together to give hydrophobic domains, play an important role in the process for modified release of the AP, just as in the stabilization of the latter.

It may be hypothesized that physical (noncovalent) interactions occur between the hydrophobic domains of the amphiphilic polymer PO and the AP (in particular protein AP). This strong affinity of the protein for the polymer results in its prolonged release after subcutaneous administration. This mechanism of release differs in particular from, and can optionally be combined with, that observed for the microparticles of polylactic acid, of polylactic-glycolic acid or also with those of sodium hyaluronate in which the release of the AP is essentially related to the diffusion of the active principle and to the decomposition/erosion of the particle.

The affinity of the protein for the polymer PO makes it possible to limit the phenomena of aggregation of the proteins and other damaging events which can occur during the atomization process, without the need to have recourse to other stabilizing agents (for example, sugars or surfactants). This protective effect of PO also makes it possible to easily obtain liquid formulations which are stable on storage. Finally, the PO, when it has an essentially amorphous nature (in particular when it is a polyamino acid), confers excellent properties of physical stability on the microparticles when they are stored in the dry form.

It is thus apparent that the presence of these amphiphilic polyamino acids PO introduces additional means for controlling the modified release of the AP and its stabilization with regard to aggregation or possible chemical decompositions.

Furthermore, the amphiphilic nature of the polymer PO makes it possible, during the atomization process, to be able to use either an entirely aqueous phase or an entirely organic volatile phase or a volatile mixture of aqueous and organic phase, which offers great flexibility in the implementation of the process.

In addition, due to their chemical nature, the microparticles formed from a PO of the (co)polyamino acid type are biodegradable, are biocompatible and generally have good properties of local tolerance.

As regards the process for producing the solid and dry microparticles according to the invention, namely atomization, it is by nature easily convertible to the industrial scale.

Finally, the microparticles according to the invention and the formulations comprising them are nontoxic and well tolerated locally.

### DETAILED DESCRIPTION OF THE INVENTION

### Definitions

Throughout this patent application, the conjunction “or” should be taken in the inclusive sense of “either or both”.

Thus, for example, a linear alkyl which can comprise at least one heteroatom (O, N or S) or at least one unsaturation can have two heteroatoms, N and S, and one unsaturation.

Throughout the present account, unlike the microparticles according to the invention, the term “submicron-scale particles” or “nanoparticles” denotes particles with a size (measured in a T test described below) of greater than or equal to 1 nm and of less than 500 nm, preferably of between 5 and 250 nm.

Within the meaning of the invention and throughout the present account, the term “polyamino acid” covers both natural polyamino acids and synthetic polyamino acids, and also oligoamino acids comprising from 10 to 20 amino acid residues in the same way as polyamino acids comprising more than 20 amino acid residues.

The amino acid residues preferred in the main polyamino acid chain are those having the L configuration; the type of bond preferred is the a type, that is to say a peptide bond between an α-amino group of an amino acid and the carboxyl group, in the 1 position, of another α-amino acid.

Within the meaning of the invention, the term “protein” denotes, for example, both a protein and a peptide, whether an oligopeptide or a polypeptide. This protein (or this peptide) may or may not be modified, for example, by grafting one or more polyoxyethylene groups.

Within the meaning of the invention and throughout the present account, the terms “association” or “to associate” employed to describe the relationships between one or more APs and the polymers POs mean in particular that the active principle or principles are bound to the polymer(s) PO(s) by a noncovalent bond, for example by electrostatic or hydrophobic interaction or hydrogen bond or steric interference.

1st Aspect of the Invention: The Microparticles

Characteristic (a):

The fact of associating, before atomization, the AP, for example a protein or another peptide compound, with an amphiphilic polymer PO, such as an amphiphilic (co)polyamino acid, exhibits numerous advantages, both with regard to the process itself and with regard to the properties of the microparticles produced by atomization.

The physical atomization treatment confers, on the dry solid microparticles obtained, a specific structure which is the source of a good number of their advantageous properties. This structure is correctly characterized by this method of production by atomization, as well as by the functions attached to these advantageous properties.

Characteristic (b):

This characteristic is defined objectively by the T test described below.

### T Test for Measuring the Size of the Microparticles by Laser Diffraction:

a—T0 Test in the Case where the Microparticles are in the Dry Form

### 1 Equipment and Operation Conditions

| | |
|---|---|
| Laser particle sizer | Malvern Mastersizer 2000 |
| Unit | Hydro 2000SM liquid route |
| Volume of the dispersing carrier fluid | 150 ml |
| Wavelengths (blue and red) | 466 and 632 nm |
| Stirring speed | 2400 rev/min |
| Analytical range | 0.02 μm to 2000 μm |
| Optical model (Mie theory) | |
| Values of the refractive indices used: | |
| Dispersing fluid (heptane) | n_{fluid} = 1.39 + i0 |
| Polystyrene latex | n_{polystyrene latex} = 1.59 + i0 |
| Values of the obscuration to trigger the | Between 5% and 20% |
| analysis | |
| Acquisition time | 10 s |

### 2 Preparation of the Sample

A 0.1% solution of Span 80 in heptane is prepared (to do this, 0.01 g of Span 80 powder is weighed out in a 20 ml flask and then heptane is added by weighing in order to obtain a final weight of 10 g),
approximately 6 mg of powder are weighed out in a 5 ml test tube,
0.7 g of heptane comprising 0.1% of Span 80 is added to the test tube,
the test tube is placed for 2 min in an ultrasonic bath in order to thoroughly disperse the powder

### 3 Analysis of the Sample

The circulating fluid stored in the liquid-route dispersing system at rest is emptied and replaced by heptane. The stirring of the Hydro 2000SM module is adjusted to 2400 rev/min.

Measuring is begun with the abovementioned experimental conditions:
Alignment of the laser beam,
Recording of the background noise.

After these stages, the sample to be analyzed is introduced in the following way: the diluted sample is added dropwise (with a Pasteur pipette) until the obscuration is between 5% and 20% and acquisition is started.

The data relating to the D50, which is the diameter below which 50% of the objects analyzed are found, are obtained.

The mean of the D50 of 3 measurements carried out on 3 different preparations is produced.

b—T1 Test in the Case where the Microparticles are in the Form of an Aqueous Dispersion

### 1 Equipment and Operation Conditions

| | |
|---|---|
| Laser particle sizer | Malvern Mastersizer 2000 |
| Unit | Hydro 2000SM liquid route |
| Volume of the dispersing carrier fluid | 150 ml |
| Wavelengths (blue and red) | 466 and 632 nm |
| Stirring speed | 2400 rev/min |
| Analytical range | 0.02 μm to 2000 μm |
| Optical model (Mie theory) | |
| Values of the refractive indices used: | |
| Dispersing fluid (water) | n_{fluid} = 1.33 + i0 |
| Polystyrene latex | n_{polystyrene latex} = 1.59 + i0 |
| Values of the obscuration to trigger the | Between 5% and 20% |
| analysis | |
| Acquisition time | 10 s |

### 2 Preparation of the Sample

The sample to be analyzed is introduced into the cell as is or can optionally be rediluted with water in the case of highly scattering samples.

### 3 Analysis of the Sample

The circulating fluid stored in the liquid-route dispersing system at rest is emptied and replaced by fresh demineralized water. The stirring of the Hydro 2000SM module is adjusted to 2400 rev/min.

Measuring is begun with the abovementioned experimental conditions:
Alignment of the laser beam,
Recording of the background noise.

After these stages, the sample to be analyzed is introduced in the following way: the diluted sample is added dropwise (with a Pasteur pipette) until the obscuration is between 5% and 20% and acquisition is started.

The data relating to the D50, which is the diameter below which 50% of the objects analyzed are found, are obtained.

The mean of the D50 of 3 measurements carried out on 3 different preparations is produced.

c—T2 Test in the Case where the Microparticles are in Dispersion in an Organic Solvent

This test is analogous to the T1 test. Nevertheless, in this case, it is necessary to replace the water by a solvent which is fully miscible with the dispersing liquid and in which the particles do not swell. In many cases, heptane is used.

### 1 Equipment and Operating Conditions

| | |
|---|---|
| Laser particle sizer | Malvern Mastersizer 2000 |
| Unit | Hydro 2000SM liquid route |
| Volume of the dispersing carrier fluid | 150 ml |
| Wavelengths (blue and red) | 466 and 632 nm |
| Stirring speed | 2000 rev/min |
| Analytical range | 0.02 μm to 2000 μm |
| Optical model (Mie theory) | |
| Values of the refractive indices used: | |
| Dispersing fluid (for example heptane) | nₕₑₚₜₐₙₑ = 1.39 + i0 |
| Polystyrene latex | n_{polystyrene latex} = 1.59 + i0 |
| Values of the obscuration to trigger the | Between 5% and 20% |
| analysis | |
| Acquisition time | 10 s |

### 2 Preparation of the Sample

In order to prepare the sample to be analyzed, 400 μl of the sample to be analyzed have to be diluted in a 5 ml test tube with 600 μl of heptane and then the preparation has to be vortexed for 10±5 s.

### 3 Analysis of the Sample

The circulating fluid stored in the liquid-route dispersing system at rest is emptied and replaced by heptane. The stirring of the Hydro 2000SM module is adjusted to 2400 rev/min.

Measuring is begun with the abovementioned experimental conditions:
Alignment of the laser beam,
Recording of the background noise.

After these stages, the sample to be analyzed is introduced in the following way: the diluted sample is added dropwise (with a Pasteur pipette) until the obscuration is between 5% and 20% and acquisition is started.

The data relating to the D50, which is the diameter below which 50% of the objects analyzed are found, are obtained.

The mean of the D50 of 3 measurements carried out on 3 different preparations is produced.

Characteristic (c):

This characteristic is defined objectively by the DP1 test described below.

DP1 Test of Dispersibility of the Powders:

Approximately 30 mg of particle powder are introduced into a 3 ml flask equipped with a septum. The exact weight of the particles is then determined (w₁). 1 ml of reconstitution solution is added to the powder through the septum using a 1 ml syringe equipped with a 25 G×⅝ (0.5×16 mm) needle (of BD Microlance™ 3 type, for example) and the flask is stirred by hand intermittently for 15 min. The flask is weighed, so as to determine the exact weight of solution introduced (w₂). At the end of this time, the entire suspension is sucked up through a 1 ml syringe (of Braun Injeckt-F Luer 1 ml type, ref. 9166017V) equipped with a 25 G needle and decanted into a fresh flask tared beforehand, and the weight w₃ of solution recovered is determined.

The qualitative aspect of the dispersion (tendency to disperse, opacity, visual homogeneity of the solution), the possibility or not of injecting it through a 25 G needle and the percentage of solution recovered:

%=W₃×100/(w₁+w₂)

are recorded and the size of the microparticles is measured according to a T1 or T2 test (depending on whether the dispersing liquid is an aqueous solution or an organic liquid).

It is considered that the powder has good dispersing properties in the liquid if:
the suspension obtained comprises microparticles with a mean diameter of between 2 and 40 μm,
the appearance of the suspension is homogeneous,
it can be injected through a 25 G needle and if
at least 80% of the suspension can thus be recovered.

Apart from the characteristics (a), (b) & (c), the microparticules according to the invention can be defined objectively by the fact that they are stable in an ST1 test or in an ST2 test.

ST1 Test of Physical Stability of the Dry Microparticles:

The size of the microparticles present in the dry atomized powder is measured within the week following atomization of the powder on a laser particle sizer according to the T0 test. The powder is then placed in an oven at 30° C. for one week. A control sample is left at 5° C. A size measurement is again carried out after dispersion under the same conditions as at time t0. The distributions of the particles before and after aging are compared.

ST2 Test of Colloidal Stability of the Particles in Suspension:

The microparticle powder is dispersed in the dispersing liquid with magnetic stirring at the concentration at which it is desired to observe its stability and is left stirring with moderate magnetic stirring for at least 2 h (measurement t0).

The size of the particles is measured on a laser particle sizer according to the T1 or T2 test according to the dispersing medium (aqueous or organic).

The suspension is subsequently left standing for 7 days at 5° C. The sedimentation pellet is dispersed by stirring for a few minutes (until a suspension is obtained which is homogeneous after visual observation). A size measurement is again carried out after dispersion under the same conditions as at time t0.

The polymer PO

The polymers POs according to the invention are water-soluble biodegradable polymers carrying hydrophobic groups HG and hydrophilic groups (preferably ionizable groups IGs which are at least partially ionized). The hydrophobic groups HG can be small in number with respect to the remainder of the chain and can be situated laterally to the chain or inserted in the chain and can be distributed randomly (random copolymer) or distributed in the form of sequences or grafts (block copolymers or sequential copolymers).

According to a preferred embodiment of the invention, the polymer PO is an amphiphilic (co)polyamino acid.

Such a choice of PO provides excellent compatibility with the APs of protein/peptide type. It is thus possible to greatly simplify the composition of the starting solution or suspension of the AP of protein/peptide type with the polymer PO. This starting solution or suspension can comprise solely the AP and the PO in an aqueous or organic phase. The absence of other ingredients makes it possible for such a starting solution or suspension to be able to be filtered (pore size: 0.2 μm) before the atomization, which makes it possible to carry out the latter under aseptic conditions.

According to a preferred embodiment of the invention, the PO is chosen from amphiphilic (co)polyamino acids and their blends.

Preferably, the polyamino acids according to the present invention are oligomers or homopolymers comprising glutamic or aspartic acid repeat residues or copolymers comprising a mixture of these two types of residues. The residues under consideration in these polymers are amino acids having the D, L or D/L configuration and are bonded via their α or γ positions for the glutamate or glutamic residue and their α or β positions for the aspartic or aspartate residue.

According to an even more preferred embodiment of the invention, the polymer PO is a polyamino acid formed by aspartic residues or glutamic residues, at least a portion of these residues carrying grafts having at least one hydrophobic group HG. These polyamino acids are in particular of the type of those disclosed in PCT patent application WO-A-00/30618.

According to a first possibility, the PO(s) is(are) defined by the following general formula (I) (the —COOR^{S} radical includes the forms where the bond between the carboxyl and R³ is an ionic bond —COO⁻⁺R³):

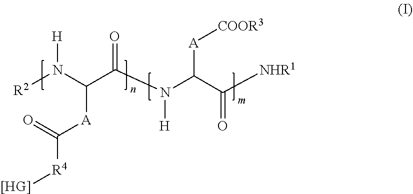

in which:
R¹ is chosen from the group consisting of H, linear C₂ to C₁₀ alkyl, branched C₃ to C₁₀ alkyl, benzyl and —R⁴-[HG], or
NHR¹ is a terminal amino acid residue;
R² is chosen from the group consisting of H, linear C₂ to C₁₀ acyl, branched C₃ to C₁₀ acyl and —R⁴-[HG] or
R² is a terminal pyroglutamate residue;
R³ is H, or
⁺R³ is preferably selected from the group consisting of:
   metal cations advantageously chosen from the subgroup consisting of: sodium, potassium, calcium and magnesium,
   organic cations advantageously chosen from the subgroup consisting of:
      amine-based cations,
      oligoamine-based cations,
      polyamine-based cations (polyethyleneimine being particularly preferred),
      cations based on amino acid(s) advantageously chosen from the class consisting of cations based on lysine or on arginine,
   or cationic polyamino acids advantageously chosen from the subgroup to consisting of polylysine or oligolysine;
R⁴ represents a direct bond or a spacer based on 1 to 4 amino acid residues;
A independently represents a —CH₂— radical (aspartic residue) or a —CH₂—CH₂— radical (glutamic residue);
n/(n+m) is defined as the molar degree of grafting and its value is sufficiently low for PO, dissolved in water at pH=7 and at 25° C., to form a colloidal suspension of submicron-sized particles of PO, preferably, n/(n+m) is between 1 and 25 mol % and better still between 1 and 15 mol %;
n+m is defined as the degree of polymerization and varies from 10 to 1000, preferably between 50 and 300;
HG represents a hydrophobic group comprising 6 to 30 carbon atoms.

In a preferred embodiment of the invention:
HG is chosen from the group consisting of alkoxy radicals of the type:
—OCH₂(CH₂—CH₂)₃₋₈—CH₃, oleyl, tocopheryl or cholesteryl, and
R⁴ represents a simple valency bond.

According to a second possibility, PO corresponds to one of the following general formulae (II), (III) and (IV) (the —COOR^{3′} radical includes the forms where the bond between the carboxyl and R^{3′} is an ionic bond —COO⁻⁺R^{3′}):

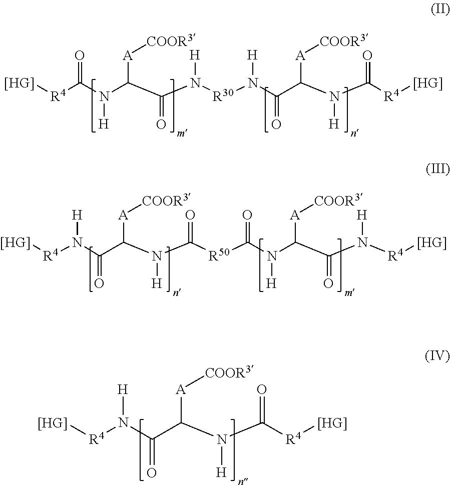

in which:
HG represents a hydrophobic group comprising 6 to 30 carbon atoms;
R³⁰ is a divalent linear C₂ to C₆ alkylene chain;
R^{3′} is H, or
⁺R^{3′} is preferably selected from the group consisting of:
   metal cations advantageously chosen from the subgroup consisting of: sodium, potassium, calcium and magnesium,
   organic cations advantageously chosen from the subgroup consisting of:
      amine-based cations,
      oligoamine-based cations,
      polyamine-based cations (polyethyleneimine being particularly preferred),
      cations based on amino acid(s) advantageously chosen from the class consisting of cations based on lysine or on arginine,
   or cationic polyamino acids advantageously chosen from the subgroup consisting of polylysine or oligolysine;
R⁵⁰ is a divalent linear C₁ to C₈ alkylene chain in which one or two methylene units, preferably at each end of R⁵⁰, can be independently replaced by —O— or —NH—;
R⁴ represents a direct bond or a spacer based on 1 to 4 amino acid residues;
A independently represents a —CH₂— radical (aspartic residue) or a —CH₂—CH₂— radical (glutamic residue);
n′+m′ or n″ is defined as the degree of polymerization and varies from 10 to 1000, preferably between 50 and 300.

According to an advantageous alternative form, R⁴ represents a simple valency bond.

According to a third possibility, PO is an essentially neutral copolyhydroxyalkylglutamine (preferably, alkyl is ethyl) comprising a multiplicity of pendant hydrophobic groups (HGs) which are identical to or different from one another. The copolyhydroxyalkylglutamine carries hydroxyalkylamino groups. These hydroxyalkylamino groups are preferably bonded to the copolymer via an amide bond. The hydroxyalkylamines which can be used to amidate the carboxyl of the glutamate residues and to give this copolyhydroxyalkylglutamine are identical to or different from one another and are, for example, chosen from the following: 2-hydroxyethylamine, 3-hydroxypropylamine, 2,3-dihydroxypropylamine, tris(hydroxymethyl)aminomethane and 6-hydroxyhexylamine.

Advantageously, at least one of the hydrophobic groups HGs is included in a hydrophobic graft comprising at least one spacing joint (or unit) (spacer) which makes it possible to connect the hydrophobic group HG to a main chain of copolyglutamates. This joint can comprise, for example, at least one direct covalent bond or at least one amide bond or at least one ester bond. For example, the joint can be of the type of those belonging to the group comprising in particular: amino acid residues, derivatives of aminoalcohols, derivatives of polyamines (for example diamines), derivatives of polyols (for example diols) and derivatives of hydroxy acids. The grafting of the HGs to the copolyglutamate or polyhydroxyalkylglutamine chain can involve the use of HG precursors capable of being bonded to the copolyglutamate or copolyhydroxyalkylglutamine chain. The precursors of the HGs are in practice, and without this being limiting, chosen from the group consisting of alcohols and amines, it being possible for these compounds to be easily functionalized by a person skilled in the art. For further details with regard to this copolyhydroxyalkylglutamine (preferably, alkyl is ethyl), reference will be made to FR-A-2,881,140.

According to an advantageous alternative form, in particular according to at least one of the three possibilities targeted above, all or part of the hydrophobic radicals HGs of the POs are chosen independently from the group of radicals consisting of:
a linear or branched alkoxy which comprises from 6 to 30 carbon atoms and which can comprise at least one heteroatom (preferably O, N or S) or at least one unsaturation,
an alkoxy comprising 6 to 30 carbon atoms and having one or more annulated cycloalkyls and optionally comprising at least one unsaturation or at least one heteroatom (preferably O, N or S),
an alkoxyaryl or an aryloxyalkyl of 7 to 30 carbon atoms which can comprise at least one unsaturation or at least one heteroatom (preferably O, N or S).
   According to another advantageous alternative form, in particular according to at least one of the three possibilities targeted above, the hydrophobic group HG is chosen from the group consisting of alkoxy radicals of the type:
—OCH₂(CH₂—CH₂)₃₋₈—CH₃, oleyl, tocopheryl or cholesteryl, and
R⁴ represents a simple valency bond.

According to another advantageous alternative form, the n HG groups of the PO, in particular according to at least one of the three possibilities targeted above, each represent, independently of one another, a monovalent radical of the following formula:

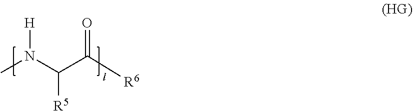

in which:
R⁵ is methyl (alanine residue), isopropyl (valine), isobutyl (leucine), sec-butyl (isoleucine) or benzyl (phenylalanine);
R⁶ represents a hydrophobic radical comprising from 6 to 30 carbon atoms;
l varies from 0 to 6.

According to a noteworthy characteristic of the invention, all or part of the hydrophobic radicals R⁶ of the POs are chosen independently from the group of radicals consisting of:
a linear or branched alkoxy which comprises from 6 to 30 carbon atoms and which can comprise at least one heteroatom (preferably O, N or S) or at least one unsaturation,
an alkoxy comprising 6 to 30 carbon atoms and having one or more annulated cycloalkyls and optionally comprising at least one unsaturation or at least one heteroatom (preferably O, N or S),
an alkoxyaryl or an aryloxyalkyl of 7 to 30 carbon atoms which can comprise at least one unsaturation or at least one heteroatom (preferably O, N or S).

In practice, and without this being limiting, the hydrophobic radical R⁶ of the graft of the PO is chosen from the group consisting of alkoxy radicals of the type:
—OCH₂(CH₂—CH₂)₃₋₈—CH₃, oleyl, tocopheryl or cholesteryl.

Advantageously, the main chain of the polyamino acid is:
[symbol] an α-L-glutamate or α-L-glutamic homopolymer;
[symbol] an α-L-aspartate or α-L-aspartic homopolymer;
[symbol] or an α-L-aspartate/α-L-glutamate or α-L-aspartic/α-L-glutamic copolymer.

In a noteworthy way, the distribution of the aspartic or glutamic residues of the main polyamino acid chain of the PO is such that the polymer thus formed is either random or of block type or of multiblock type.

According to another method of definition, PO has a molar mass which lies between 2000 and 100 000 g/mol and preferably between 5000 and 40 000 g/mol.

According to an alternative form, PO carries at least one graft of polyalkylene glycol type bonded to a glutamate or aspartate residue.

Advantageously, this graft of polyalkylene glycol type is of following formula (V):

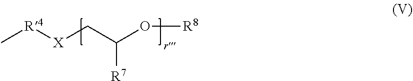

in which:
R′⁴ represents a direct bond or a “spacer” based on 1 to 4 amino acid residues;
X is a heteroatom chosen from the group consisting of oxygen, nitrogen and sulfur;
R⁷ and R⁸ independently represent an H or a linear C₁ to C₄ alkyl;
n′″ varies from 10 to 1000, preferably from 50 to 300.

In practice, the polyalkylene glycol is, for example, a polyethylene glycol.

It is desirable, in accordance with the invention, for the molar percentage of grafting to the polyalkylene glycol to vary from 1 to 30%.

In addition, polyamino acids POs are extremely advantageous owing to the fact that, with an adjustable degree of grafting, they are dispersed in water at pH=7.4 (for example with a phosphate buffer) to give colloidal suspensions.

Furthermore, active principles APs, such as proteins, peptides or small molecules, can join together spontaneously with nanoparticles of polyamino acids POs.

It should be understood that the POs comprise ionizable groups which are, depending on the pH and the composition, either neutral (for example, COOH) or ionized (for example COO). For this reason, the solubility in an aqueous phase is directly a function of the fraction of ionized functional groups and thus of the pH. In aqueous solution, in the case of carboxyl functional groups, the counterion can be a metal cation, such as sodium, calcium or magnesium, or an organic cation, such as the protonated forms of triethanolamine, of tris(hydroxymethyl)aminomethane or of a polyamine, such as polyethyleneimine.

The POs of polyamino acid type are obtained, for example, by methods known to a person skilled in the art. Random polyamino acids can be obtained by grafting the hydrophobic graft, functionalized beforehand by the spacer, directly to the polymer by a conventional coupling reaction. Block or multiblock polyamino acids POs can be obtained by sequential polymerization of the corresponding N-carboxyamino acid anhydrides (NCA).

A polyamino acid which is a homopolyglutamate, a homopolyaspartate or a block, multiblock or random glutamate/aspartate copolymer is prepared, for example, according to conventional methods.

The most widely used technique for obtaining polyamino acid of a type is based on the polymerization of N-carboxyamino acid anhydrides (NCA) described, for example, in the paper “Biopolymers, 1976, 15, 1869” and in the work by H. R. Kricheldorf, “alpha-Amino acid N-carboxy Anhydride and related Heterocycles”, Springer-Verlag (1987). When the side chain possesses a carboxylic acid functional group, the NCA derivatives are preferably NCA-O-Me, NCA-O-Et or NCA-O-Bz esters (Me=methyl, Et=ethyl and Bz=benzyl). The polymers are subsequently hydrolyzed under conditions appropriate for obtaining the polymer in its acid form. These methods are inspired by the description given in application FR-A-2 801 226 of the Applicant Company. A certain number of polymers which can be used according to the invention, for example of poly(α-L-aspartic), poly(α-L-glutamic), poly(α-D-glutamic) and poly(γ-L-glutamic) type with variable weights are available commercially. The polyaspartic of α,β type is obtained by condensation of aspartic acid (to obtain a polysuccinimide), followed by basic hydrolysis (cf. Tomida et al., Polymer 1997, 38, 4733-36).

The coupling of the graft with an acid functional group of the polymer is carried out easily by reaction of the polyamino acid in the presence of a carbodiimide as coupling agent and optionally a catalyst, such as 4-dimethylaminopyridine, and in an appropriate solvent, such as dimethylformamide (DMF), N-methylpyrrolidone (NMP) or dimethyl sulfoxide (DMSO). The carbodiimide is, for example, dicyclohexylcarbodiimide or diisopropylcarbodiimide. The degree of grafting is controlled chemically by the stoichiometry of the constituents and reactants or the reaction time. The hydrophobic grafts functionalized by a spacer are obtained by conventional peptide coupling or by direct condensation by acid catalysis. These techniques are well known to a person skilled in the art.

NCA derivatives synthesized beforehand with the hydrophobic graft are used for the synthesis of block or multiblock copolymer. For example, the NCA-hydrophobe derivative is copolymerized with the NCA-O-Bz and then the benzyl groups are selectively removed by hydrolysis.

The Active Principle(s) PA(s)

As regards the AP, it is preferably chosen from the group consisting of: proteins, glycoproteins, proteins bonded to one or more polyalkylene glycol chains [preferably polyethylene glycol (PEG): “PEGylated proteins”], peptides, polysaccharides, liposaccharides, oligonucleotides, polynucleotides and their mixtures, and, more preferably still, from the subgroup of erythropoietin, oxytocin, vasopressin, adrenocorticotropic hormone, epidermal growth factor, platelet-derived growth factor (PDGF), hematopoietic growth factors and their mixtures, factors VIII and IX, hemoglobins, cytochromes, albumins, prolactin, luteinizing hormone-releasing hormone (LHRH), LHRH antagonists, LHRH agonists, human, porcine or bovine growth hormones (GHs), growth hormone-releasing hormone, insulin, somatostatin, glucagon, interleukins or their mixtures (IL-2, IL-11, IL-12), interferons-α, -β or -γ, gastrin, tetragastrin, pentagastrin, urogastrone, secretin, calcitonin, enkephalins, endorphins, angiotensins, thyrotropin-releasing hormone (TRH), tumor necrosis factor (TNF), neurotrophic factors (NGF), granulocyte growth factor (G-CSF), ganulocyte-macrophage growth factor (GM-CSF), macrophage growth factor (M-CSF), heparinase, bone morphogenetic protein (BMP), atrial natriuretic peptide (hANP), glucagon-like peptide 1 (GLP-1), vascular endothelial growth factor (VEGF), recombinant hepatitis B surface antigen (rHBs), renin, cytokines, bradykinin, bacitracins, polymyxins, colistins, tyrocidine, gramicidins, cyclosporins and synthetic analogs, and pharmaceutically active modifications and fragments of enzymes, of cytokines, of antibodies, of antigens and of vaccines.

According to an alternative form, the AP is a hydrophobic, hydrophilic or amphiphilic small organic molecule of the type of those belonging to the family of the anthracyclines, taxoids or camptothecins or of the type of those belonging to the family of the peptides, such as leuprolide or cyclosporin, and their mixtures. Within the meaning of the present account, a small molecule is in particular a small nonprotein molecule, for example devoid of amino acids.

According to another alternative form, the AP is advantageously chosen from at least one of the following families of active substances: agents for the treatment of alcohol abuse, agents for the treatment of Alzheimer's disease, anesthetics, agents for the treatment of acromegaly, analgesics, antiasthmatics, agents for the treatment of allergies, anticancer agents, antiinflammatories, anticoagulants and antithrombotics, anticonvulsants, antiepileptics, antidiabetics, antiemetics, antiglaucomas, antihistaminics, antiinfectives, antibiotics, antifungals, antivirals, antiparkinsonians, anticholinergics, antitussives, carbonic anhydrase inhibitors, cardiovascular agents, hypolipidemics, antiarrythmics, vasodilators, antianginals, antihypertensives, vasoprotectants, cholinesterase inhibitors, agents for the treatment of disorders of the central nervous system, stimulants of the central nervous system, contraceptives, fertility promoters, inducers and inhibitors of uterine labor, agents for the treatment of mucoviscidosis, dopamine receptor agonists, agents for the treatment of endometriosis, agents for the treatment of erectile dysfunctions, agents for the treatment of fertility, agents for the treatment of gastrointestinal disorders, immunomodulators and immunosuppressants, agents for the treatment of memory disorders, antimigraines, muscle relaxants, nucleoside analogs, agents for the treatment of osteoporosis, parasympathomimetics, prostaglandins, psychotherapeutic agents, sedatives, hypnotics and tranquilizers, neuroleptics, anxiolytics, psychostimulants, antidepressants, agents for dermatological treatments, steroids and hormones, amphetamines, anorectics, nonanalgesic painkillers, barbiturates, benzodiazepines, laxatives, psychotropics and any combination of these products.

Quantitatively, it is particularly advantageous for the fraction by weight of AP not associated with the micron-scale particles [nonassociated AP] as % by weight to be such that:
[nonassociated AP]=1
preferably [nonassociated AP]=0.5.

2^{nd} Aspect of the Invention: The Process for Producing the Microparticles by Atomization of a Solution or Colloidal Suspension of PO Comprising AP

According to a preferred form of the process according to the invention, the PO present in the solution or colloidal suspension intended to be atomized is at least partly in the form of PO nanoparticles having a size, measured in the T1 test, of less than 500 nm, preferably of between 10 and 300 nm and more preferably still of between 10 and 100 nm.

Advantageously, the concentration of PO in the solution or colloidal suspension to be atomized is, for example, between 5 mg/ml and 100 mg/ml, preferably between 10 mg/ml and 40 mg/ml.

In the context of the invention, the AP/PO association is produced before or during the atomization stage.

For this association, the PO or the AP can be in the solid form (preferably a powder) or in the form of a liquid suspension.

Techniques for associating one or more APs with PO before the atomization stage are described in particular in patent application WO-A-00/30618. They consist, for example, in mixing a colloidal suspension of PO with a solution or suspension of AP. According to one alternative form, the AP in the powder form is mixed with the PO suspension.

According to an advantageous alternative form of this production process, the microparticles of polymer PO associated with at least one active principle (AP) are dispersed in an essentially aqueous liquid medium, said medium preferably comprising a dispersing means M1, and the dispersion obtained is lyophilized.

The lyophilizate thus obtained has the advantage of facilitating the preparation of the liquid formulations based on the microparticles (3rd aspect of the invention described below) as this lyophilizate rapidly disperses in the reconstituting liquid of use in preparing abovesaid liquid formulations.

Advantageously, the dispersing means M1 is chosen from the group consisting of:
i—polyvalent ions, the polarity of which is opposed to the polarity of the ionizable groups of the polymer PO and which are present in the aqueous continuous phase;
ii—at least one hydrophilic compound (preferably which can be used for an injectable preparation) added to the PO suspension/solution to be atomized and thus present in the atomized PO/AP microparticles;
iii—at least one coating of the microparticles with at least one film of at least one hydrophilic compound (preferably which can be used for an injectable preparation);
iv—the pH change;
v—and the combinations of at least two of the means (i) to (iv);
the means (i) being particularly preferred.

For further details with regard to examples of implementational possibilities of the means M1 (i) to (iv), reference will be made to the description which follows of the dispersing means M2.

Likewise, as regards the liquid dispersing medium, the latter can comprise the same additives as those described for the reconstituting liquid defined below.

3^{rd} Aspect of the Invention: Liquid Formulations Based on the Microparticles Obtained by Atomization of a Solution or Colloidal Suspension of PO Comprising AP

The formulation according to the invention can be a liquid pharmaceutical form for the prolonged release of AP comprising a colloidal suspension, of low viscosity, based on PO microparticles associated with at least one AP, these microparticles being those as defined above or those obtained by the process itself also as defined above and in the examples below.

This formulation has the advantage of being injectable parenterally and of being liquid under the injection conditions.

According to the invention, the qualifiers “low” or “very low” viscosity advantageously correspond to a dynamic viscosity at 20° C. of less than or equal to 1000 mPa·s. Preferably, the dynamic viscosity of the formulation, measured at 20° C., for a shear gradient of 1000 s⁻¹, is preferably less than or equal to 500 mPa·s, preferably between 2 and 200 mPa·s, for example between 1.0 and 100 mPa·s, indeed even between 1.0 and 50 mPa·s.

Measurement of the Dynamic Viscosity

The reference measurement for the dynamic viscosity can be carried out, for example, at 20° C. using an AR1000 rheometer (TA Instruments) equipped with cone/plate geometry (4 cm, 2°). The viscosity v is measured for a shear gradient of 10 s⁻¹.

This low viscosity renders the formulations of the invention easily injectable parenterally, in particular by the mucosal, subcutaneous, intramuscular, intradermal, intraperitoneal or intracerebral route or into a tumor. The formulation according to the invention can also be administered by the oral, nasal, pulmonary, vaginal or ocular route, inter alia.

The viscosity of the liquid formulations of the invention is low, both at injection temperatures corresponding to ambient temperatures, for example between 4 and 30° C., and at the physiological temperature.

The dry microparticles formed by atomization of amphiphilic PO (for example polyamino acid) can furthermore be easily redispersed to form microparticle suspensions or solutions of low viscosity.

Dispersing Means M2

As regards specifically the dispersing or suspending of the atomized PO/AP microparticles in a reconstituting liquid in order to prepare the formulation, the invention provides for said formulation to comprise a means for dispersing the PO/AP microparticles.

This dispersing means M2 can differ according to the nature of the continuous phase (that is to say, of the reconstituting liquid) of the suspension used to form the formulation.

Thus, four possibilities can be envisaged in particular according to the invention:
1. The continuous phase of the suspension is essentially aqueous.
2. The continuous phase of the suspension is an essentially organic water-miscible phase.
3. The continuous phase of the suspension is an essentially organic water-immiscible phase.
4. The continuous phase of the suspension is an essentially organic water-miscible or water-immiscible phase.

The term “essentially aqueous continuous phase” is understood to mean, for example, a liquid comprising at least 60% by weight of water.

The term “essentially organic continuous phase” is understood to mean, for example, a liquid comprising at least 60% by weight of organic phase.

1. The Continuous Phase of the Suspension is Essentially Aqueous

Several types of dispersing means M2, optionally combined with one another, can be envisaged, namely that the dispersing means M2 is, for example, chosen from the group consisting of:
i—polyvalent ions, the polarity of which is opposed to the polarity of the ionizable groups of the polymer PO and which are present in the aqueous continuous phase;
ii—at least one hydrophilic compound (preferably which can be used for an injectable preparation) added to the PO suspension/solution to be atomized and thus present in the atomized PO/AP microparticles;
iii—at least one coating of the microparticles with at least one film of at least one hydrophilic compound (preferably which can be used for an injectable preparation);
iv—the pH change;
v—and the combinations of at least two of the means (i) to (iv); the means (i) being particularly preferred.

(i) Dispersing Means M2 Based on Polyvalent Ions

In the case where PO exhibits ionizable groups IGs, the dispersing means M2 can comprise polyvalent ions, the polarity of which is the opposite of the polarity of the ionizable groups IGs (at least partially ionized) of the polymer PO, which are introduced into the aqueous continuous phase during the reconstitution of the suspension/solution which forms the suspension.

Within the meaning of the invention, the term “polyvalent ions” denotes, for example, divalent ions, trivalent ions, tetravalent ions and mixtures of these ions.

In the case where PO exhibits anionic IG groups, the polyvalent ions are polyvalent cations, preferably divalent cations, more preferably still chosen from the group consisting of: Mg²⁺, Ca²⁺, Zn²⁺, Fe²⁺, Cu²⁺ and their mixtures, or trivalent cations, more preferably still chosen from the group consisting of: Al³⁺, Fe³⁺ and their mixtures.

It is possible for the formulation according to the invention, in addition to polyvalent ions, to comprise monovalent ions (for example cations), which may be active in the aggregation of the nanoparticles to give microparticles.

These polyvalent ions are introduced into the formulation of the invention preferably in the form of an aqueous salt (organic or inorganic) solution, for example a solution of sulfate, chloride, acetate, gluconate or glutamate (or other anionic amino acid) of polyvalent cations.

This dispersing means M2 based on polyvalent ions is more preferably suitable in the case where amphiphilic PO (for example a (co)polyamino acid) is relatively hydrophilic.

(ii) and (iii) Dispersing Means M2 Based on Hydrophilic Compound(s) or Comprising at Least One Coating Based on Hydrophilic Compound(s)

The dispersing means M2 can comprise essentially:
at least one hydrophilic compound (preferably which can be used for an injectable preparation) added to the PO suspension/solution to be atomized and thus present in the atomized PO/AP microparticles;
or at least one coating of the microparticles with at least one film of at least one hydrophilic compound (preferably which can be used for an injectable preparation).

These dispersing means M2 can be incorporated in the formulation according to several methods.

According to a first method, at least one hydrophilic compound chosen from those which can be used in an injectable preparation is added to the PO suspension/solution to be atomized, this hydrophilic compound preferably being chosen from the group consisting of:
amino acids (for example lysine, arginine, glycine);
polyalkylene glycols, preferably polyethylene glycols;
copolyalkylene glycols, preferably ethylene glycol/propylene glycol copolymers (of Poloxamer, Pluronic or Lutrol type);
cellulose polymers and their derivatives, preferably carboxyalkylcelluloses (for example carboxymethylcelluloses) or alkylcelluloses (for example methylcelluloses);
hydrogenated or nonhydrogenated saccharides, such as trehalose, sorbitol, mannitol or sucrose;
polyols, such as propylene glycol or glycerol;
gelatins, preferably hydrolyzed gelatins;
nitrogenous (co)polymers, preferably those present in the group consisting of polyacrylamides, poly(N-vinylamide)s, polyvinylpyrrolidones (PVPs) and poly(N-vinyllactam)s;
polyvinyl alcohol)s (PVAs);
poly(sodiumglutamate);
and their mixtures;
   said hydrophilic coating compound preferably comprising at least one hydrophilic polymer.

According to a second method, the microparticles are coated with at least one layer of at least one hydrophilic compound as defined above.

In this second method, the hydrophilic compound preferably comprises at least one hydrophilic polymer chosen from the hydrophilic compounds as defined above.

The dispersing means M2 (ii) based on at least one hydrophilic compound has proven to be particularly suitable for amphiphilic POs exhibiting a sufficiently high hydrophobicity, for example a level of HG groups of greater than or equal to 10 mol % for a PO composed of at least one polyamino acid.

The coating [means M2 (iii)] of the microparticles with such a layer of hydrophilic and biocompatible compound can be carried out, for example, by two successive atomizations; the second atomization, carried out on a suspension of particles in a solvent immiscible with said microparticles, can contribute to facilitating the dispersing of these particles.

This dispersing means M2 (iii) by hydrophilic coating of the microparticles allows the suspension of microparticles to remain reliable and stable at least for a few days, which makes it easier to handle.

Advantageously, the combination of a means M2 (ii) or M2 (iii) for dispersing the microparticles based on hydrophilic compound(s) and of a means M2 (i) for dispersing based on divalent ions present in the aqueous phase during the reconstitution of the formulation makes possible accelerated dispersing.

According to another specific embodiment, the aqueous continuous phase or the hydrophilic coating can also comprise at least one injectable surfactant, such as Tween® 80, a lecithin, phosphatidylethanolamine, phosphatidylserine or a polyoxypropylene/polyoxyethylene copolymer (trade name: Poloxamer, Pluronic, Lutrol).

(Iv) Dispersing Means by pH Change

Another dispersing means which can be envisaged according to the invention consists of a dispersing means by pH change, preferably before atomization. This type of means has proven to be suitable in particular in the case where the amphiphilic PO (for example polyamino acid) is a compound carrying ionizable functional groups which is furthermore highly hydrophilic, that is to say comprising, for example, less than 10 mol % of hydrophobic groups HGs.

This is because the use of such a means M2 (iv) makes it possible to increase the hydrophobicity of the amphiphilic PO by taking the pH to a value such that the ionizable functional groups of the PO (for example of the polyamino acid) become nonionized (for example, acidification in the case where the PO is a hydrophilic polyamino acid of the polyGlu or polyAsp type).

Preferably, this dispersing means M2 (iv) by pH change before atomization is applied to the PO suspension/solution immediately before atomization.

This dispersing means M2 (iv) by pH change before atomization may or may not be combined:
with the dispersing means M2 (i) based on polyvalent ions employed after atomization, in the reconstitution stage; or
with the dispersing means M2 (ii) or (iii) based on hydrophilic compound, preferably by coating M2 (iii) the microparticles with at least one hydrophilic polymer.

2. The Continuous Phase of the Suspension is an Essentially Organic Water-Miscible Phase

The dispersing means consists of this water-miscible organic phase. This phase can thus, for example, comprise ethanol, N-methylpyrrolidone, dimethyl sulfoxide, isopropanol, glycerol or glycofurol (tetrahydrofurfuryl alcohol polyethylene glycol ether).

3. The Continuous Phase of the Suspension is an Essentially Organic Water-Immiscible Phase

According to an advantageous embodiment, the dispersing means comprises a lipophilic liquid, the melting point of which is preferably less than or equal to 15° C., present in the water-immiscible organic continuous phase.

Preferably, the lipophilic liquid comprises:
a mixture of triglycerides of saturated C₈-C₁₀ fatty acids resulting from coconut oil, for example that sold under the name Miglyol 812 by Sasol;
at least one vegetable oil, preferably soybean oil, palm oil, linseed oil, cottonseed oil, sesame oil, sunflower oil or peanut oil;
at least one lipid, preferably a liquid lecithin, synthetic or natural vitamin E;
at least one lipid derivative, preferably arachidonylphosphatidylcholine and stearoylphosphatidylcholine,
at least one fatty acid, preferably oleic acid, myristic acid, palmitic acid, stearic acid and their salts;
at least one fatty acid derivative, preferably a mono-, di- or triglyceride derivative, ethyl oleate, lauryl lactate, glyceryl stearate, sorbitan palmitate, sorbitan stearate, sorbitan monooleate or polysorbate;
and their mixtures;
   with the condition according to which, in the case where some of the products listed above taken separately are not liquid at a temperature of less than or equal, for example, to 15° C., then these products are mixed with others so that they are liquid at a temperature of less than or equal, for example, to 15° C.

Derivatives of fatty acid triglycerides are particularly preferred, in particular a mixture of triglycerides of saturated C₈-C₁₀ fatty acids resulting from coconut oil, for example that sold under the name Miglyol 812 by Sasol. Other triglycerides comprising long chains of fatty acids which can be used are present in particular in vegetable oils, such as soybean oil, palm oil, linseed oil, cottonseed oil, sesame oil, sunflower oil or peanut oil.

The atomized PO/AP powders are easily dispersed in this water-immiscible organic phase to give stable suspensions of microparticles which remain whole for several days and are thus here again easy to handle. This dispersing is particularly rapid without it being necessary to add another or other dispersing means, even if this possibility can be envisaged.

4. The Continuous Phase of the Suspension is an Essentially Organic Water-Miscible or Water-Immiscible Phase

According to an advantageous processing alternative compatible with the 2nd & 3rd possibilities regarding the nature of the continuous phase of the suspension/solution constituting the formulation, the dispersing means comprises a coating of the microparticles with at least one film-forming coating compound (preferably which can be used for an injectable preparation).

Preferably, the film-forming coating compound comprises at least one hydrophobic polymer chosen from the group consisting of polylactides, polyglycolides, poly(lactide-co-glycolide)s, polyorthoesters, polyanhydrides, poly(hydroxybutyric acid)s, polycaprolactones, poly(alkyl carbonate)s, water-insoluble PO polymers, their derivatives and their blends.

According to one alternative form, the film-forming coating compound is of lipid nature and exhibits a melting point preferably of greater than or equal to 15° C. and comprises at least one lipid or at least one lipid derivative, or at least one vegetable oil, or at least one fatty acid or at least one fatty acid derivative or at least one mixture of triglycerides of saturated fatty acids.

According to another advantageous embodiment, the organic continuous phase, for example hydrophobic, or the hydrophobic coating can also comprise at least one injectable surfactant, such as Tween® 80, a lecithin, phosphatidylethanolamine, phosphatidylserine or a polyoxypropylene/polyoxyethylene copolymer (trade name: poloxamer, Pluronic, Lutrol).

Excipients/Stabilizers

It can also be advantageous, to facilitate the properties of dispersing in the aqueous phase or to further improve the stability of the aqueous suspensions, for the injectable formulation to comprise other additives, on the one hand, those denoted below by “excipients/stabilizers” and, on the other hand, conventional excipients.

The excipients/stabilizers can be selected from the group consisting of:
nanoparticles of at least one polymer PO, PO being a water-soluble biodegradable amphiphilic copolymer carrying hydrophobic groups (HGs) and at least partially ionized ionizable hydrophilic groups (IGs) which spontaneously forms a colloidal suspension of nanoparticles in water, at pH=7.0, under isotonic conditions;
amino acids;
polyalkylene glycols, preferably polyethylene glycols;
copolyalkylene glycols, preferably ethylene glycol/propylene glycol copolymers (of Poloxamer, Pluronic or Lutrol type);
cellulose polymers and their derivatives, preferably carboxyalkylcelluloses (for example carboxymethylcelluloses) or alkylcelluloses (for example methylcelluloses);
esters of sorbitan and of fatty acid(s), preferably esters of polyoxyalkylene (for example ethylene) glycol and of at least one acid (for example oleic acid), of Tween® (or polysorbate) type;
surfactants based on phospholipids and on polyalkylene glycols, preferably on polyethylene glycols;
hydrogenated or nonhydrogenated saccharides, such as trehalose, sorbitol, mannitol or sucrose;
polyols, such as propylene glycol or glycerol;
gelatins, preferably hydrolyzed gelatins;
nitrogenous (co)polymers, preferably from the group consisting of polyacrylamides, poly(N-vinylamide)s, polyvinylpyrrolidones (PVPs) and poly(N-vinyllactam)s;
poly(vinyl alcohol)s (PVAs);
and their mixtures.

One of the preferred excipients (stabilizers) in accordance with the invention is formed by nanoparticles of at least one polymer PO which is identical to or different from (preferably identical to) that constituting the microparticles. The amount of excipient/stabilizer employed in the formulation is preferably between 0.01 and 10% by weight and more preferably still between 0.1 and 5% by weight. As regards the stabilizers comprising nanoparticles, they are advantageously used in the formulation in a proportion of 1.5 to 3.5% by weight, for example of 2.0 to 3.0 (≈2.5)% by weight.

The Reconstituting Liquid

The reconstituting liquid employed in the preparation of the formulation according to the invention can comprise, for example:
at least one buffer or at least one injectable salt (phosphate buffer, citrate buffer, sodium chloride) at a concentration, for example, between 0.001M and 0.1M, preferably between 0.005M and 0.02M, this buffer or this injectable salt making it possible to adjust the pH of the solution;
at least one injectable surfactant, preferably of polysorbate type, such as Tween® 20 and Tween® 80, or of poloxamer type, such as Lutrol® F38, Lutrol® F68 or Lutrol® F 127, in concentrations, for example, of between 0.01% and 2%, preferably between 0.05 and 0.5%.

The reconstituting liquid can additionally comprise densifying agents, such as saccharides, namely, for example, sucrose, D-mannitol or trehalose, in concentrations of between 0.1% and 10%, preferably between 0.5 and 5%. The reconstituting solution can also comprise an injectable viscosifying polymer chosen from the group consisting of polysaccharides, synthetic polymers (for example sodium carboxymethylcellulose), poly(vinyl alcohol), polyvinylpyrrolidone, polyalkylene glycols (for example polyethylene glycols), and their blends.

The use of dry microparticles of amphiphilic PO (for example polyamino acid) and the use of an ad hoc reconstituting means thus makes it possible to meet the twofold objective, which is to be able to obtain pharmaceutical formulations which are both stable and easily dispersible in order to allow them to be injected parenterally.

In addition to the excipients/stabilizers targeted above for improving the dispersing and the stability, the other conventional excipients which can be added to the suspension/solution to be atomized or to the formulation according to the invention are, for example, antimicrobials, buffers, antioxidants or agents which make it possible to adjust the isotonicity which are known to a person skilled in the art. Reference may be made, for example, to the work: Injectable Drug Development, P. K. Gupta et al., Interpharm Press, Denver, Colo., 1999.

This addition of conventional excipients can be carried out either in the aqueous dispersing phase or in the solution/suspension before atomization.

Application of the Liquid Formulation According to the Invention

Although the formulation according to the invention is preferably a pharmaceutical formulation, this does not, however, exclude cosmetic, health food or plant protection formulations comprising at least one PO as defined above and at least one AP.

4th Aspect of the Invention: Kit for Reconstituting the Formulation According to the Invention

The PO/AP microparticles and the essentially aqueous reconstituting liquids, the essentially organic water-miscible reconstituting liquids and the essentially organic water-immiscible reconstituting liquids are defined above.

5th Aspect of the Invention: Reconstituting Process, in Particular for Reconstituting the Formulation According to the Invention

The PO/AP microparticles and the essentially aqueous reconstituting liquids, the essentially organic water-miscible reconstituting liquids and the essentially organic water-immiscible reconstituting liquids are defined above.

In accordance with the invention, it is possible to envisage providing sterilizing filtration, through filters with a porosity equal, for example, to 0.2 μm, of the liquid suspension of nanoparticles from which the micron-scale particles of the formulation according to the invention result. The aggregation under sterile conditions according to the method of preparation described above thus makes it possible to directly inject the formulation into a patient.

6th Aspect of the Invention: Solid Pharmaceutical Formulation

This formulation for the prolonged release of AP comprises a dry powder form for inhalation and pulmonary administration based on PO/AP microparticles as defined above.

Other Aspects of the Invention

The present invention is also targeted at solid products derived from the PO microparticles according to the invention.

In practice, these derived products can be composed in particular of powders, gels, implants or films, inter alia.

Thus, the invention is targeted at products derived from the formulation according to the invention, taken as such, whatever their process of preparation.

The invention also relates to a therapeutic treatment method consisting essentially in administering an effective amount from a therapeutic standpoint of the formulation as described in the present account by the parenteral route, in particular by the mucosal, subcutaneous, intramuscular, intradermal, intraperitoneal or intracerebral route or into a tumor. The formulation according to the invention can also be administered by the oral, nasal, pulmonary, vaginal or ocular route, inter alia.

According to a specific alternative form of the invention, this therapeutic treatment method consists in administering the formulation as described above by parenteral, subcutaneous, intramuscular, intradermal, intraperitoneal or intracerebral injection or injection into a tumor, preferably so that it forms a deposited layer at the injection site.

The invention will be better understood and its advantages and alternative embodiments will clearly emerge from the examples which follow and which describe the synthesis of the POs formed by polyamino acids grafted by a hydrophobic group, their conversion to a system for prolonged release of AP, namely to a formulation according to the invention (powder of dry microparticles), and the stability and redispersibility characteristics of such systems.

### EXAMPLES

### Example 1

### Synthesis of an Amphiphilic Polymer PO-A

### Synthesis of a Polyglutamate Grafted by α-Tocopherol of Synthetic Origin

15 g of a poly(α-L-glutamic acid) (with a weight equivalent to approximately 16 900 Da with respect to a polyoxyethylene standard and obtained by polymerization of NCAGluOMe, followed by hydrolysis, as are disclosed in application FR-A-2 801 226) are dissolved in 288 ml of dimethylformamide (DMF) by heating at 80° C. until the polymer has dissolved. The solution is cooled to 15° C. and 2.5 g of D,L-α-tocopherol (>98%, obtained from Fluka®), dissolved beforehand in 8 ml of DMF, 280 mg of 4-dimethylaminopyridine, dissolved beforehand in 1 ml of DMF, and 1.6 g of diisopropylcarbodiimide, dissolved beforehand in 6 ml of DMF, are successively added. After stirring for 3 h, the reaction medium is poured into 1200 ml of water comprising 15% of sodium chloride and hydrochloric acid (pH=2). The precipitated polymer is subsequently recovered by filtration and washed with 0.1N hydrochloric acid, with water and with diisopropyl ether. The polymer is subsequently dried in an oven under vacuum at 40° C. A yield of the order of 90% is obtained. The molar mass, measured by steric exclusion chromatography, is 15 500 with respect to a polyoxyethylene standard. The level of grafted tocopherol, estimated by proton NMR spectroscopy, is 5.1 mol %. A suspension of nanoparticles of polymer in water is obtained by dissolving it in water and by adjusting the pH to 7±1 (neutralization of the carboxylates).

### Example 2

### Synthesis of an Amphiphilic Polymer PO-B

Example 2 is adapted from example 1, a degree of grafting of 20% being targeted.

### Example 3

### Preparation of Dry Micron-Scale Particles of Polymer PO-A Comprising IFN-α2B

Preparation of a Solution Comprising 20 mg/g of Polymer and 0.25 mg/g of IFN

200 g of 30 mg/g solution of polymer PO-A are introduced into a 500 ml flask. 68 g of water are subsequently introduced into the flask comprising the polymer. A frozen solution of IFN-α-2b concentrated to 2.8 mg/g is defrosted at 25° C. for 1 h and 26.8 g of this defrosted solution are introduced into the flask comprising the polymer solution. The mixture is left at ambient temperature for 14 h.

The solution is filtered through a 0.2 μm sterilizing filter.

### Atomization of the Polymer-IFN Solution

The solution is atomized on a spray-drying device of Büchi B290 type. The liquid solution is sucked up at a rate of 5 ml/min and nebulized through a spray nozzle fed with nitrogen (700 kPa, 900 l/h). The suction flow rate (drying air) is 40 m³/h. The inlet temperature is maintained at 90° C., which results, under these conditions, in an outlet temperature of 45° C.

### Characterization of the Microparticles Obtained:

The size of the particles obtained (according to T0 test) is: D50=5 μm.

After dispersing the powder in water at the atomization concentration, the IFN assay by HPLC is identical to that of the solution before atomization. No decomposed form is detected.

### Example 4

### Preparation of Dry Micron-Scale Particles of Polymer PO-A Comprising IFN-α2B and Polysorbate 80

The solution of polymer PO and of interferon is prepared in an identical way to example 3. 0.9 g of polysorbate 80 is added to the solution before atomization.

### Atomization of the Polymer-IFN Solution in the Presence of Polysorbate

The solution is atomized under conditions identical to those described in example 3.

### Characterization of the Microparticles Obtained:

The size of the particles obtained (according to T0 test) is: D50=5 μm.

After dispersing the powder in water at the atomization concentration, the IFN assay by HPLC is identical to that of the solution before atomization. No decomposed form is detected.

### Example 5

### Preparation of Acidified Dry Micron-Scale Particles of Polymer PO-A Comprising IFN-α2B

The solution of polymer PO-A and of IFN is prepared in an identical way to example 3. It is subsequently diluted by addition of water so that the concentration of polymer PO-A is 15 mg/g (the IFN concentration then being 0.188 mg/g). After filtering through a 0.2 μm filter, the solution is acidified by addition of 1N HCl until a pH=4 is obtained. The solution thus obtained is opalescent.

### Atomization of the Acidified Polymer-IFN Solution

The solution is atomized under conditions identical to those described in example 3.

### Characterization of the Microparticles Obtained:

The size of the particles obtained (according to T0 test) is: D50=5 μm.

After dispersing the powder in water at the atomization concentration, the IFN assay by HPLC is identical to that of the solution before atomization. No decomposed form is detected.

### Example 6

### Preparation of Dry Micron-Scale Particles of Polymer PO-A which are Coated with PVP and which Comprise IFN-α2B

A powder formed of dry microparticles of polymer PO-A and of IFN is obtained from a PO-A/IFN mixture according to the protocol used in example 3.

On conclusion of the atomization stage, 1.5 g of this powder are resuspended in an ethanolic solution comprising 7 g/l of polyvinylpyrrolidone (PVP) of injectable grade of PVP K30 type. The ethanolic suspension is stirred for 2 h and then it is atomized a second time on the spray-drying device of Büchi B290 type equipped with an extraction trap for organic solvent (inert loop at −20° C.) and operating as a closed circuit under nitrogen. The liquid solution is sucked up at a rate of 5 ml/min and nebulized through a spray nozzle fed with nitrogen (700 kPa-670 l/h). The suction flow rate (drying air) is 40 m³/h. The inlet temperature is maintained at 80° C., which results, under these conditions, in an outlet temperature of 55° C.

### Characterization of the Microparticles Obtained:

The size of the particles obtained (according to T0 test) is: D50=6 μm. After dispersing the powder in water at the atomization concentration, the IFN assay by HPLC is identical to that of the solution before atomization. No decomposed form is detected.

### Example 7

### Preparation of Dry Micron-Scale Particles of Polymer PO-B Comprising IFN-α2B

The protocol for preparing the polymer-IFN solution and for atomizing is identical to that described in example 3 but replacing the polymer PO-A by the polymer PO-B.

### Characterization of the Microparticles Obtained:

The size of the particles obtained (according to T0 test) is: D50=4 μm.

After dispersing the powder in water at the atomization concentration, the IFN assay by HPLC is identical to that of the solution before atomization. No decomposed form is detected.

### Example 8

### Preparation of Dry Micron-Scale Particles of Polymer PO-A Comprising hGH

### Preparation of the Concentrated hGH Solution:

60 g of a solution of recombinant human growth hormone (concentration 3.9 mg/g) are defrosted at 25° C. for 90 min and concentrated approximately 6 times by frontal ultrafiltration through a membrane with an exclusion limit of 10 kD until a concentration of 24 mg/g is obtained (monitored by HPLC).

Mixture with the Polymer Solution

52 g of a concentrated 30 mg/g solution of polymer PO-A are diluted to 19.5 mg/g by addition of 28 g of water. 8 g of the 24 mg/g hGH solution are slowly poured onto the polymer solution thus diluted. The mixture is left overnight at ambient temperature and then filtered through a sterilizing filter (pore size: 0.2 μm).

### Atomization of the Polymer/hGH Solution

The solution is atomized under conditions identical to those described in example 3.

### Characterization of the Microparticles Obtained:

The size of the particles obtained (according to T0 test) is: D50=5 μm.

After dispersing the powder in water at the atomization concentration, the hGH assay by HPLC is identical to that of the solution before atomization. No decomposed form is detected.

### Example 9

### Preparation of Dry Micron-Scale Particles of Polymer PO-A Comprising IL-2

### Preparation of the Concentrated IL-2 Solution:

100 g of a frozen solution of IL-2 at the concentration of 2 g/l stabilized by SDS are defrosted at ambient temperature and then the solution is cooled to a temperature of between 0 and 2° C. 100 g of absolute ethanol are slowly added to this solution so as to precipitate the protein. The precipitate is recovered by filtration through a 0.65/0.45 μm membrane in a frontal diafiltration cell and washed with 200 g of water. The precipitate is dried by application of a stream of nitrogen. The precipitate is then dissolved in 10 g of precooled (<5° C.) 0.02N sodium hydroxide so as to obtain a clear solution of protein at 20 mg/g and pH=12 devoid of SDS. The exact assay of the solution is determined by an HPLC method.

Mixture with the Polymer Solution

96 g of a concentrated solution of polymer PO of example 1 (at 30 mg/g) are diluted by addition of 39 g of water. 9 g of the preceding 20 mg/g concentrated IL-2 solution are slowly poured onto the polymer solution thus diluted.

The mixture comprising 20 mg/g of polymer PO and 1.25 mg/g of IL-2 is left overnight at ambient temperature and then filtered through a 0.2 μm sterilizing filter.

### Atomization of the Polymer-IL-2 Solution

The solution is atomized under conditions identical to those described in example 3.

### Characterization of the Microparticles Obtained:

The size of the particles obtained (according to T0 test) is: D50=5 μm.

After dispersing the powder in water at the atomization concentration, the IL-2 assay by HPLC is identical to that of the solution before atomization. No decomposed form is detected.

### Example 10

### Preparation of Dry Micron-Scale Particles of Polymer PO-A Comprising Insulin

Preparation of 40 g of a 20.3 mg/g Concentrated Insulin Solution:

0.83 g of recombinant human insulin (powder) with an activity of 28.7 UI/g and with a water content of 2.5% is introduced into a glass flask. 23.62 g of water are added and the insulin is dispersed with slow magnetic stirring. 6.22 g of 0.1N HCl are added until a clear acidic insulin solution is obtained. 9.32 g of 0.1N sodium hydroxide are then added so as to obtain a clear final solution having a pH of between 7 and 8. The insulin solution is filtered through a 0.2 μm sterilizing filter.

Mixture with the Polymer Solution

37.66 g of the preceding concentrated insulin solution are slowly poured (with magnetic stirring) onto 220 g of 30 mg/g polymer PO solution. 72.34 g of water are added to the solution. The mixture is left at ambient temperature for 4 h and then filtered through a 0.2 μm filter.

### Atomization of the Polymer-Insulin Solution

The solution is subsequently atomized on a spray-drying device of the Büchi B290 type. The liquid solution is sucked up at a rate of 5 ml/min and nebulized through a spray nozzle fed with nitrogen (700 kPa-900 l/h). The suction flow rate (drying air) is 40 m³/h. The inlet temperature is maintained at 120° C., which results, under these conditions, in an outlet temperature of 70° C.

### Atomization of the Polymer-Insulin Solution

The solution is atomized under conditions identical to those described in example 3.

### Characterization of the Microparticles Obtained:

The size of the particles obtained (according to T0 test) is: D50=5 μm.

After dispersing the powder in water at the atomization concentration, the insulin assay by HPLC is identical to that of the solution before atomization. No decomposed form is detected.

### Example 11

### Characteristics of the Microparticles Obtained According to the Invention: Size and Stability

The size of the microparticles is measured according to the T0 test and the stability according to the S1 test. The decomposition of the protein is evaluated by HPLC by comparing a chromatogram of the formulation before and after atomization.

| | D50 | D50 size after | Decomposition |
|---|---|---|---|
| | size according | subjecting to stability | of the protein |
| | to T0 | according to ST1 | brought about |
| Examples | (μm) | (μm) | by atomization* |
| 3 | 5 | 5 | none |
| 4 | 5 | 5 | none |
| 5 | 5 | 5 | none |
| 6 | 6 | 5 | none |
| 7 | 4 | 5 | none |
| 8 | 5 | 5 | none |
| 9 | 5 | 5 | none |
| 10 | 5 | 5 | none |

*“None” means that no decomposed form has been observed.

The results demonstrate that the atomization of the proteins in the presence of an amphiphilic polyamino acid makes it possible to retain the stability of the protein. In the solid form, the powder is stable according to the protocol described. It is an inevitable condition and it is anticipated that the stability of the powders is at least 2 years at 5° C.

Atomization is a method capable of decomposing the protein. The analysis by HPLC shows that no form of decomposition is observed on comparing the chromatograms before and after atomization.

The atomization of a protein in the presence of an amphiphilic polyamino acid results in microparticles which are stable over time and does not cause decomposition of the protein.

These powders can be used in a solid form (inhalation, needleless injection by a gun under pressure, for example) or in the form of an injectable suspension after reconstitution in an appropriate medium.

### Example 12

### Reconstitution of Suspension Starting from the Powders of Examples 3 to 10

The powders are reconstituted in three different media according to the protocol described for carrying out the DP 1 test:
A. An aqueous phosphate buffer saline solution at pH 7.4
B. A 0.1M aqueous magnesium chloride solution
C. A capric acid triglyceride solution (Miglyol® 812)

The samples are subsequently evaluated by carrying out the following stages:
dispersing in one of the media mentioned above
transferring into a syringe and then injecting through a 25 G needle.

The suspension is considered to have good properties if at least 80% is recovered by suction/injection through a 25 G needle.

### Results:

| | Medium A | | | | | |
|---|---|---|---|---|---|---|
| Ex- | | ST2 | Medium B | | Medium C | |
| am- | DP1 | sta- | DP1 | ST2 | DP1 | ST2 |
| ples | test | bility | test | stability | test | stability |
| 3 | Does not | | Conforms | Conforms | Conforms | Conforms |
| | conform | | | | | |
| 4 | Does not | | Conforms | Conforms | Conforms | Conforms |
| | conform | | | | | |
| 5 | Does not | | Does not | | Conforms | Conforms |
| | conform | | conform | | | |
| 6 | Does not | | Conforms | Conforms | Conforms | Conforms |
| | conform | | | | | |
| 7 | Conforms | Con- | Conforms | Conforms | Conforms | Conforms |
| | | forms | | | | |
| 8 | Does not | | Conforms | Conforms | Conforms | Conforms |
| | conform | | | | | |
| 9 | Does not | | Conforms | Conforms | Conforms | Conforms |
| | conform | | | | | |
| 10 | Does not | | Conforms | Conforms | Conforms | Conforms |
| | conform | | | | | |

It has also been measured that the protein is not decomposed after reconstitution in the media A, B and C during the stability test.

These results show that only with the composition of Example 7 is it possible to obtain a stable and injectable suspension of the microparticles in water buffered by PBS at pH=7.4. For the majority of the examples (except 5), the reconstituting medium comprising a divalent salt makes it possible to improve these properties. In Miglyol medium, all the formulations are easily dispersible, injectable and stable.

The combination of these properties is not obtained easily according to the polymer used. It is to the credit of the inventors to have found, after numerous trials, that certain excipients and reconstituting media make it possible to obtain these properties.

### Example 13

### Measure of Viscosity of the Reconstituting Formulations

A 30 mg/ml dispersion of the powders in the media B and C of the preceding examples (except 5 in the medium B) give stable suspensions of low viscosity; in the medium B, the viscosities measured are all less than 10 mPa·s and, in the medium C, the viscosities are all of the order of 30 to 40 mPa·s.

By way of comparison, a composition of the same polymers in the nanoparticulate form according to the prior art cannot be obtained at this concentration (30 mg/ml) with acceptable viscosity values (<100 mPa·s).

### Example 14

### Preparation of Dry Micron-Scale Particles of Polymer PO-A Comprising IFN-α2B Obtained in the Form of a Lyophilized Powder

The particles are first of all prepared as described in example 3. The atomization stage is carried out under aseptic conditions and the particles are recovered in a sterile container.

### Redispersion of the Particles in the Presence of Mg²⁺ Ions:

Immediately after the atomization phase, the particles are recovered and redispersed under aseptic conditions in a 0.10M aqueous MgCl₂ solution. The amount of MgCl₂ solution added is adjusted so that the concentration of polymer PO-A in the suspension is approximately 40 mg/ml. The pH is adjusted to 6.5 by addition of 1N sodium hydroxide solution.

### Lyophilization

The suspension is distributed in containers of Lyoguard® type which make it possible to keep the suspension sterile during lyophilization: the containers are subsequently lyophilized under sterile conditions during a 72 h cycle on a laboratory freeze dryer (benchtop module, Christ, Osterode, Germany). The powder is distributed under sterile conditions in flasks before use.

### Example 15

### Comparison of the Reconstitution of the Powders Formed of Microparticles Obtained from Example 3 and Those from Example 14

For this comparison, the volumes of suspension reconstituted are identical in both cases (approximately 1 ml) and the reconstituting flasks are identical (3 ml glass flasks). The amounts of powder are adjusted so that the two suspensions comprise, at the end, 40 mg/ml of polymer and 0.5 mg/ml of IFN-α2B. The powder from example 3 is reconstituted in a 0.1M aqueous MgCl₂ solution while the powder from example 14 (which already comprises Mg²⁺ ions) is reconstituted in pure water.

In a first step, the flasks are stirred manually. While the powder from example 3 requires at least 15 min in order to be redispersed, the dispersing is faster for the powder from example 14 and a homogeneous suspension is obtained in less than 5 min. A magnetic bar is then inserted into the flasks and the two flasks are stirred in identical fashion for 1 h. At the end of this stirring, the characteristics of the two suspensions are compared: the sizes of the particles and the viscosities of the two suspensions are similar.

| | | Characteristics after | |
|---|---|---|---|
| | | 1 h of magnetic stirring | |
| | Rate of redispersion | | Viscosity |
| | with manual stirring | D50 (T₁ test) | (20° C., 1000 s⁻¹) |
| Powder from | approximately | 12 μm | 54 mPa · s |
| example 3 | 15 min | | |
| Powder from | <5 min | 10 μm | 53 mPa · s |
| example 14 | | | |

### Example 16

### Pharmacokinetics of IFN in the Dog after Subcutaneous Injection of a Formulation Based on Amphiphilic Polyamino Acids in the Microparticulate Form

Eight naïve Beagle dogs (weight of 9±0.6 kg) were treated with the following formulations:

| | | | | | | Dose |
|---|---|---|---|---|---|---|
| | Number | [IFN] | [PO] | pH/ | Dose | volume |
| Formulation | of dogs | (mg/ml) | (mg/ml) | mOsm | (μg/kg) | (ml/kg) |
| IFN IR | 4 | 0.5 | 0 | 6.5/354 | 60 | 0.12 |
| Formulation 1 | 4 | 0.5 | 40 | 6.5/560 | 60 | 0.12 |

The IFN IR (IR for immediate release) corresponds to a solution of recombinant human interferon (PCGen, batch IB05.0516) adjusted in concentration, pH and osmolarity ([IFN]=0.5 mg/ml, pH=6.5 and 354 mOsm).

The particles of formulation 1 are prepared according to example 14 from the same batch of PCGen interferon: the lyophilized powder is resuspended in water under sterile conditions (under a laminar flow hood) according to a process analogous to that described in example 15.

The pharmacokinetic results are combined in the following table:

| | Cₘₐₓ ± SD | T > 50 pg/ml ± SD | AUC₀₋ₐₗₗ ± SD | RBA | T_{50%AUC} ± SD |
|---|---|---|---|---|---|
| Formulation | (ng/ml) | (h) | (ng · h/ml) | (%) | (h) |
| IFN IR | 25.2 ± 0.4 | 22.6 ± 0.6 | 162.5 ± 27.2 | 100 | 5.1 ± 0.7 |
| Formulation 1 | 0.5 ± 0.2 | 225.0 ± 15.3 | 54.7 ± 7.5 | 34 | 99.5 ± 11.9 |

where:
Cₘₐₓ is the maximum serum concentration of IFN;
T > 50 pg/ml is the time where the serum concentration of IFN is greater than 50 pg/ml;
AUC represents the area under the curve for serum concentration of IFN as a function of time;
RBA represents the bioavailability with respect to an Immediate Release formulation;
T_{50%AUC} represents the time necessary to release 50% of the IFN released in total.

The IFN IR exhibits a rapid release profile with a maximum serum concentration of 25.2±0.4 ng/ml, achieved after a median time of 5 h (range: 3-5 h). The circulating IFN can no longer be quantified beyond 24 h.

Formulation 1 presents a major modification to the pharmacokinetic profile of IFN, with a very slow release and a maximum serum concentration of 0.5±0.2 ng/ml (50 times lower than that of the IR form) achieved after a median time of 60 h (range: 48-96 h). The general appearance of the pharmacokinetics is a pseudoplateau flat profile. The level of circulating IFN returns to a nonquantifiable concentration between 192 h and 240 h (8 and 10 days). This formulation exhibits a lower AUC: relative bioavailability loss of 66% (RBA=34%). The T_{50%AUC} is approximately 20 times greater than that of the IFN IR.

### Example 17

### Pharmacodynamics of Insulin in the Dog after Subcutaneous Injection of a Formulation Based on Amphiphilic Polyamino Acids in the Microparticulate Form

The reference of this example, Lantus®, is a modified insulin analog (insulin glargine—Sanofi-Aventis). The modification of two amino acids on the primary structure of human insulin confers, on Lantus®, properties of prolonged release over a period of 24 h with precipitation in situ.

A group of 6 healthy Beagle dogs (weight of 11.4±1 kg) was treated with the Lantus® formulation during a two-period crossover trial and a group of 8 healthy Beagle dogs (weight of 11.8±1 kg) was treated with formulation 2, in pairs, during a four-period crossover trial. The table in which the treatments are summarized is given below:

| | Number of | [Insulin] | [PO] | Dose | Dose volume |
|---|---|---|---|---|---|
| | dogs | (IU/g) | (mg/g) | (IU/kg) | (μl/kg) |
| Lantus ® | 12 | 100 | 0 | 1 | 10 |
| (batch 40N300) | | | | | |
| Formulation 2 | 8 | 100 | 30 | 1 | 10 |

The particles of formulation 2 are prepared according to example 10 while targeting a ratio of 30 mg of PO polymer per 3.5 mg (100 IU) of insulin. The formulation is prepared by dispersing the atomized powder in 0.1M MgCl₂ and magnetically stirring the suspension for 1 h. The pH of the formulation is 6.2 and the osmolality is 348 mOsm. The size of the corresponding particles, measured in a T₁ test, is: D50=12 μm, and the viscosity of the formulation is approximately 5 mPa·s at 20° C.

The glycemia is assayed enzymatically (hexokinase) on an automated device for biochemical analyses of the blood (Advia 1650, Bayer Diagnostics).

The pharmacodynamic results are analyzed with regard to the percentage of the basal glycemia as a function of time.

The pharmacodynamic data are combined in the following table:

| | Cₘᵢₙ ± | APGC_{0-36 h} ± | APGC_{formulation 1}/ | T_{50% APGC} ± |
|---|---|---|---|---|
| | SD | SD | APGC_{Lantus} ± SD | SD |
| | (%) | (% · h) | (%) | (h) |
| Lantus ® | 40 ± 6 | 1250 ± 342 | — | 13.3 ± 3.1 |
| Formulation | 50 ± 17 | 948 ± 491 | 76 ± 39 | 16.5 ± 6.2 |
| 2 | | | | |

where:
Cₘᵢₙ is the minimum percentage observed of the basal glycemia;
APGC_{0-36 h} (Area Percent Glycemia Curve) represents the area between the basal glycemia (100%) and the curve representing the change in the glycemia (expressed as % of the basal glycemia) over time between 0 and 36 h postdose;
T_{50% APGC} represents the time necessary to obtain 50% of the APGC_{0-36 h}.

The administration of the reference Lantus® results in a rapid fall in the glycemia from the first hour. The hypoglycemic action of the insulin glargine is subsequently maintained over a period of between 18 and 36 h (return of the glycemia to its basal level after 30 h on average).

In comparison, the administration of formulation 2 also results in a rapid fall in the glycemia from the 1st hour. The percentage of the basal glycemia is subsequently maintained at a plateau up to 36 h on average. The Cₘᵢₙ obtained with formulation 2 is on average higher than that of the reference Lantus®, which should make it possible to greatly reduce the phenomena of severe hypoglycemia in diabetic patients.

The duration of action of formulation 2 is markedly greater than that of the long-acting reference Lantus®. This is illustrated by a mean value of T_{50%APGC} which is higher for formulation 2.

A loss in APGC₀₋₃₆ₕ of only 24% was observed for formulation 2 with respect to the Lantus® reference.

What is claimed is:

1. A liquid pharmaceutical formulation for the prolonged release of AP, comprising:
microparticles of polymer (PO), obtained by atomization of an aqueous solution or colloidal suspension of PO noncovalently associated with at least one active principle (AP), the PO being a (co)polyamino acid comprising a main chain formed by aspartic or glutamic units or a mixture thereof, part of said units carrying hydrophobic groups (HG), and
an aqueous liquid comprising polyvalent cations.

2. The liquid pharmaceutical formulation of claim 1, wherein the polyvalent cations are selected from the group consisting of Mg²⁺, Ca²⁺, Zn²⁺, Fe²⁺, Cu²⁺, Al³⁺, Fe³⁺ and mixtures thereof.

3. The liquid pharmaceutical formulation of claim 1, wherein the polyvalent cations are Mg²⁺.

4. The liquid pharmaceutical formulation of claim 1, wherein said microparticles have a size comprised between 0.5 and 100 microns.

5. The liquid pharmaceutical formulation of claim 1, wherein said microparticles have a size comprised between 1 and 70 microns.

6. The liquid pharmaceutical formulation of claim 1, further comprising a at least one means for dispersing the PO microparticles, selected from:
a hydrophilic compound present in the suspension or colloidal solution of PO prior to atomization, and
a film of hydrophilic compound coated onto the PO microparticles.

7. The liquid pharmaceutical formulation of claim 6, wherein the hydrophilic compound is chosen from the group comprising:
amino acids;
polyalkylene glycols;
copolyalkylene glycols;
cellulose polymers;
hydrogenated or nonhydrogenated saccharides;
polyols;
gelatins;
nitrogenous (co)polymers;
poly(vinyl alcohol)s (PVAs);
poly(sodium glutamate);
and their mixtures.

8. The liquid pharmaceutical formulation of claim 6, wherein the hydrophilic compound is polyvinylpyrrolidone.

9. The liquid pharmaceutical formulation of claim 1, wherein PO is defined by the following general formula (I), the radical —COOR³ including forms wherein the bond between the carboxylic and R³ is a ionic bond —COO⁻⁺R³: 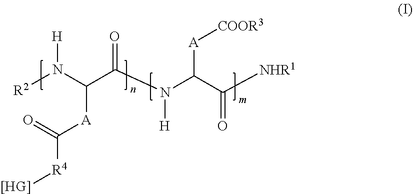
in which:
R¹ represents an H, a linear C₂ to C₁₀ or branched C₃ to C₁₀ alkyl, a benzyl, or —R⁴-[HG];
NHR¹ is a terminal amino acid residue
R² represents an H, a linear C₂ to C₁₀ or branched C₃ to C₁₀ acyl group, or —R⁴-[HG] or a terminal pyroglutamate residue;
R³ is an H, or
⁺R³ is selected from the group comprising:
metal cations,
organic cations,
or cationic polyamino acids;
R⁴ represents a direct bond or a spacer based on 1 to 4 amino acid residues;
A independently represents a —CH₂— radical or a —CH₂—CH₂— radical;
n/(n+m) is defined as the molar grafting rate and its value is sufficiently low for PO, dissolved in water at pH=7 and at 25° C., to form a colloidal suspension of submicron-sized particles of PO;
n/(n+m) is comprised between 1 and 25 mol %;
n+m is defined as the degree of polymerization and varies from 10 to 1000;
HG represents a hydrophobic group selected from the group consisting of: —OCH₂(CH₂—CH₂)₃₋₈—CH₃, oleyl, tocopheryl and cholesteryl.

10. The liquid pharmaceutical formulation of claim 9, wherein the molar grafting rate n/(n+m) is comprised between 10 and 25 mol %.

11. The liquid pharmaceutical formulation of claim 1, wherein the PO corresponds to one of the following general formulae (II), (III) and (IV), the radical —COOR^{3′} including forms wherein the bond between the carboxylic and R^{3′} is a ionic bond —COO⁻⁺R^{3′}: 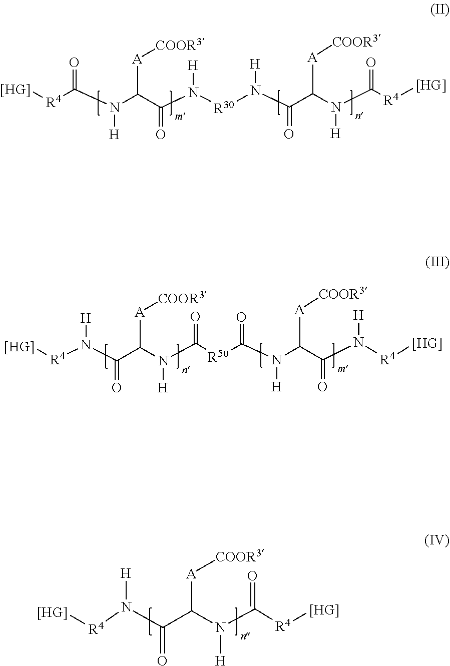
in which:
HG represents a hydrophobic group selected from the group consisting of:
—OCH₂(CH₂—CH₂)₃₋₈—CH₃, oleyl, tocopheryl and cholesteryl;
R³⁰ is a linear bivalent C₂ to C₆ alkylene chain;
R^{3′} is an H or,
⁺R^{3′} is selected from the group comprising:
metal cations,
organic cations,
or cationic polyamino acids;
R⁵⁰ is a divalent linear C₁ to C₈ alkylene chain in which one or two methylene units can be independently replaced by —O— or —NH—;
R⁴ represents a direct bond or a spacer based on 1 to 4 amino acid residues;
A independently represents a —CH₂— radical or a —CH₂—CH₂— radical;
n′+m′ or n″ is defined as the degree of polymerization and varies from 10 to 1000.

12. The liquid pharmaceutical formulation of claim 1, wherein the PO comprises at least one neutral copolyhydroxyalkylglutamine comprising a multiplicity of pendant hydrophobic groups (HGs) which are identical to or different from one another.

13. The liquid pharmaceutical formulation of claim 1, wherein the main chain of the (co)polyamino acid is selected from the group consisting of:
an α-L-glutamate or α-L-glutamic homopolymer,
an α-L-aspartate or α-L-aspartic homopolymer and
an α-L-aspartate/α-L-glutamate or α-L-aspartic/α-L-glutamic copolymer.

14. A process for the preparation of a liquid pharmaceutical formulation as claimed in claim 1, comprising the following steps:
mixing a solution of polymer (PO) with at least one AP, at pH=7.0, under isotonic conditions, the PO being a (co)polyamino acid comprising a main chain formed by aspartic or glutamic units or a mixture thereof, part of said units carrying hydrophobic groups (HG), in order to form an aqueous solution or colloidal suspension;
atomizing said colloidal suspension of PO to obtain dry solid microparticles, and
dispersing the obtained microparticles in an aqueous liquid comprising polyvalent cations.

15. The process of claim 14, comprising one additional step comprising:
adding at least a hydrophilic compound in the PO suspension or colloidal solution of PO prior to atomization.

16. The process of claim 14, comprising one additional step comprising:
coating the PO microparticles with at least a film of hydrophilic compound.

17. A process for the preparation of a solid pharmaceutical formulation, comprising the implementation of the process according to claim 14 and a further step comprising:
lyophilizing the dispersion to obtain a dry powder of microparticles.

18. A process for the preparation of a solid pharmaceutical formulation, comprising the implementation of the process according to claim 15 and a further step comprising:
lyophilizing the dispersion to obtain a dry powder of microparticles.

19. A process for the preparation of a solid pharmaceutical formulation, comprising the implementation of the process according to any of claim 16 and a further step comprising:
lyophilizing the dispersion to obtain a dry powder of microparticles.

20. A process for the preparation of a liquid pharmaceutical formulation, comprising a further step comprising:
dispersing the lyophilizate obtained by the process of claim 17 in an aqueous liquid.

21. A process for the preparation of a liquid pharmaceutical formulation, comprising a further step comprising:
dispersing the lyophilizate obtained by the process of claim 18 in an aqueous liquid.

22. A process for the preparation of a liquid pharmaceutical formulation, comprising a further step comprising:
dispersing the lyophilizate obtained by the process of claim 19 in an aqueous liquid.

23. A solid pharmaceutical formulation for the release of AP, comprising a dry powder form obtained by the process of claim 17.

24. A solid pharmaceutical formulation for the release of AP, comprising a dry powder form obtained by the process of claim 18.

25. A solid pharmaceutical formulation for the release of AP, comprising a dry powder form obtained by the process of claim 19.
